(19) United States
(12) Patent Application Publication
(10) Pub. No.: US 2024/0182833 A1
(43) Pub. Date: Jun. 6, 2024

(54) SYSTEMS AND METHODS FOR RECOVERING TARGET BIOMOLECULES FROM FIXED BED BIOREACTORS

(71) Applicant: UNIVERCELLS TECHNOLOGIES S.A., Nivelles (BE)

(72) Inventors: Jean-Christophe Drugmand, Wavre (BE); Antoine Hubert, Nivelles (BE); Tania Pereira Chilima, Nivelles (BE); Romane Meerts, Nivelles (BE)

(21) Appl. No.: 18/512,262

(22) Filed: Nov. 17, 2023

Related U.S. Application Data
(60) Provisional application No. 63/456,991, filed on Apr. 4, 2023.
(60) Provisional application No. 63/440,582, filed on Jan. 23, 2023.
(60) Provisional application No. 63/426,518, filed on Nov. 18, 2022.

(51) Int. Cl. C12M 3/06; C12M 3/00; C12N 1/06; C12N 9/22

(52) U.S. Cl. CPC C12M 27/16; C12M 23/46; C12M 23/48; C12N 1/06; C12N 9/22; C12N 2527/00

(57) ABSTRACT

The current invention relates to a method for obtaining one or more target biomolecules from cells cultured in a fixed bed bioreactor, said method comprises: the addition of a lysis solution to the bioreactor, thereby lysing the cells in the bioreactor, and prior to, simultaneous with, and/or after lysing said cells, applying one or more mechanical actions to said bioreactor, after which the one or more target biomolecules are recovered from said bioreactor. The invention further relates to a system for obtaining one or more target biomolecules from cells cultured in a fixed bed bioreactor.

This application claims the benefit of U.S. Provisional Patent Application. Ser. No. 63/456,991, filed Apr. 4, 2023, U.S. Provisional Patent Application. Ser. No. 63/440,582, filed Jan. 23, 2023, and U.S. Provisional Patent Application. Ser. No. 63/426,518, filed Nov. 18, 2022, the disclosure of which is incorporated herein by reference.

### FIELD OF THE INVENTION

The following applications are incorporated herein by reference in their entirety:

Cell Growth Matrix, PCT/EP2017/078775, 9 Nov. 2017 and published as WO2018/087235A1, 17 May 2018.

Bioreactor and Related Methods, PCT/EP2018/086394, 20 Dec. 2018 and published as WO2019/122239, 27 Jun. 2019.

Bioreactor System with Enhanced Cell Harvesting Capabilities and Related Methods, PCT/EP2022/065264, 3 Jun. 2022 and published as WO2022/254039 28 Dec. 2022.

Woven cell culture substrates, PCT/US2020/016576, 2020 Feb. 4 and published as WO2020163329A1, 2020 Aug. 13.

The systems, compositions and methods described herein relate to cell culture of adherent or suspension cells in fixed bed bioreactors, preferably in structured fixed bed bioreactors such as the bioreactors described in the patent applications listed above which are incorporated herein by reference in their entireties; and recovery of one or more target biomolecules from a cell culture harvest of the fixed bed bioreactor.

PCT publication WO2022/254039 is incorporated herein by reference in its entirety and discloses bioreactor and fixed bed configurations as well as systems and processes for applying mechanical energy to the bioreactor and fixed bed systems described therein combined with chemical/enzymatic agents. References made herein to bioreactors and fixed bed systems as well as systems for applying mechanical energy to bioreactors and fixed bed systems include but are not limited to those described therein.

The present invention relates to methods for obtaining viral vector and other target biomolecules from fixed bed bioreactors. In a second aspect, the present invention relates to a system for obtaining one or more target biomolecules from cells cultured in a fixed bed bioreactor. As such, the present invention pertains to the technical field of harvesting of target biomolecules from cells cultured in a fixed bed bioreactor.

### BACKGROUND

Fixed bed bioreactors are efficient tools to produce biological molecules such as recombinant proteins, monoclonal antibodies, viral vectors, viruses and cellular vesicles (such as exosomes). Animal cells can be used to produce such biological molecules, using for instance constitutive expression systems, infection or transfection. The target biomolecule can then be released by the cells (such as secreted recombinant proteins, “secreted” viruses or lytic viruses) or can remain intracellular (e.g. intracellular proteins, viruses or some viral vectors). Extracellular target biomolecules may be recovered in the supernatant of the bioreactor and the recirculation/perfusion loop(s), using one or more emptying and rinsing steps to recover the target biomolecule of interest (referred to herein as target biomolecule). Intracellular and partially intracellular products however remain in the cells and require lysing the cells at the end of the process using chemical/enzymatic solutions. However, common lysing conditions are quite harsh and using them can cause damage to the target biomolecule, as they are often sensitive to pH, shear stress, enzyme activity and detergents. Furthermore, target biomolecules can become trapped by cell debris or other biomolecules (e.g. cell membranes, DNA, etc.) that remain blocked inside the fixed bed structure.

The present invention aims to resolve at least some of the problems and disadvantages mentioned above. The aim of the invention is to provide a method and a system which eliminates those disadvantages and increases the recovery of intracellular (or partially intracellular) target biomolecules from fixed bed bioreactors.

### SUMMARY OF THE INVENTION

The present invention and embodiments thereof serve to provide a solution to one or more of the above-mentioned disadvantages. To this end, the present invention relates to a method for obtaining one or more target biomolecules from cells cultured in a fixed bed bioreactor according to claim 1. More particularly, the method as described herein relates to a method for obtaining one or more target biomolecules from cells cultured in a fixed bed bioreactor, said method comprises:
the addition of a lysis solution to the bioreactor, thereby lysing the cells in the bioreactor, and prior to, simultaneous with, and/or after lysing said cells, applying one or more mechanical actions to said bioreactor, after which the one or more target biomolecules are recovered from said bioreactor.

By combining a lysis solution with one or more mechanical actions, a higher yield of target biomolecules can be obtained from the bioreactor.

Preferred embodiments of the method are shown in any of the claims 2 to 11.

In a second aspect, the present invention relates to a system according to claim 12. More particularly, the system as described herein relates to a system comprising: an agitation device and a fixation system, said
agitation device comprising a deck adapted for receiving a bioreactor; and said fixation system
comprising at least one fastener for securing the bioreactor to the deck, and a bridge structure adapted for mounting to the bioreactor and receiving the at least one fastener.

Preferred embodiments of the system are shown in any of the claims 13 to 15.

In an embodiment, the invention relates to a system according to claim 12, wherein said fastener comprises an adjustable strap and wherein said fixation system further comprises one or more strap guides for locating and guiding the placement of the straps.

In an embodiment, the invention relates to a system according to any of claims 12-13, wherein the fixation system includes an annular portion part for engaging a lid or cover of the bioreactor.

In an embodiment, the invention relates to a system according to any of the previous claims 12-14, wherein the agitation device comprises a vibrating table, said vibrating table including a placeholder on which the bioreactor is to be placed.

In an embodiment, the bridge structure comprises a top portion and a depending portion.

In an embodiment, the fixation system comprises a releasable coupling.

In an embodiment, the agitation device comprises an agitator in the form of a vibrational table, a vortex device, a shaker, or another device for applying mechanical energy to the bioreactor and/or the fixed bed material.

In an embodiment the system forms part of a docking station for the bioreactor.

In an embodiment, the system further comprises a controller for controlling the agitation device.

In an embodiment, the system further comprises a controller for controlling the agitation device and the bioreactor or cell harvest processes.

In an embodiment, the bioreactor comprises a structured fixed bed bioreactor.

In an embodiment, the fixed bed comprises a plurality of cell immobilization layers.

In an embodiment, the plurality of cell immobilization layers are arranged in a stack or a spiral configuration.

In an embodiment, the cell immobilization layers are arranged either in direct contact or with a spacing between adjacent layers.

In an embodiment, the cell immobilization layers are arranged either in direct contact or with one or more spacer layers between said one or more cell immobilization layers.

In an embodiment, the fixed bed is a 3D printed fixed bed.

In an embodiment, the invention relates to a system for the recovery of biomolecules from a bioreactor, said system comprising:
a bioreactor residing on a deck of an agitating device; and
a fixation system for coupling at least a portion of the bioreactor to the agitating device.

The invention is summarized as follows:
1. A process for recovering a target biomolecule from cells in a fixed bed bioreactor, the process comprising the following steps,
draining cell culture medium from the bioreactor;
adding a rinsing solution to the bioreactor;
draining the rinsing solution from the bioreactor;
lysing the cells in the bioreactor by adding a lysis solution to the bioreactor;
draining the lysis solution from the bioreactor;
washing the cells in the bioreactor by adding a washing solution to the bioreactor,
draining the washing solution from the bioreactor; and
recovering the target biomolecule from the fixed bed bioreactor,
wherein the process further comprises adding a high conductivity solution to the bioreactor during the lysing step and/or the washing step.
2. A process for recovering a target biomolecule from cells in a fixed bed bioreactor, the process comprising the following steps,
draining cell culture medium from the bioreactor;
adding a rinsing solution to the bioreactor;
draining the rinsing solution from the bioreactor;
lysing the cells in the bioreactor by adding a lysis solution to the bioreactor;
draining the lysis solution from the bioreactor;
washing the cells in the bioreactor by adding a washing solution to the bioreactor,
draining the washing solution from the bioreactor; and
wherein the process further comprises adding a high conductivity solution to the bioreactor during the lysing step and/or the washing step.
3. The process according to embodiment 2, further comprising recovering the target biomolecule from one or more of the rinsing solution, the lysis solution and the washing solution.
4. The process according to embodiment 1 or 2, wherein the lysis solution comprises addition of a DNase, preferably a endonuclease and more preferably benzonase.
5. The process according to embodiment 1 or 2, wherein the lysis solution is low conductivity.
6. The process according to embodiment 1 or 2, wherein the conductivity of the lysis solution is increased during the lysing step.
7. The process according to embodiment 1 or 2, wherein the conductivity of the lysis solution is increased up to 1M by addition of NaCl.
8. A process for recovering a target biomolecule from cells in a fixed bed bioreactor, the process comprising one or more of the following steps:
   Perform several cycles with several rinses.
   Have sufficient contact time.
   Using DNase to degrade the free DNA
   Using surfactant to limit the adsorption of AAV to plastic vessel walls
   Use pH below the isoelectric point (pH 3 to 5) or above (pH 8 to 9).
   Applying vibrations to the bioreactor.
   Applying vibrations to the bioreactor during one or more of the lysis step and/or following rinsing step.
   Applying vibrations to the bioreactor during one or more of the processes listed hereinabove.
   Use of electrophoresis to extract one or more charged biological molecules from a fixed bed bioreactor.
   Operating one or more of the steps above in recirculation mode, preferably using a recirculation loop.
9. A process for recovering a cell or a target biomolecule from a cell in a cell culture harvest said process comprising, moving a fixed bed in the cell culture harvest; and recovering the target biomolecule from the cell culture harvest.
10. A process for recovering a target biomolecule from a fixed bed bioreactor comprising cells said process comprising, moving the fixed bed; and recovering the target biomolecule.
11. A process for recovering a target biomolecule from a fixed bed bioreactor comprising cells in a solution said process comprising, moving the solution relative to the fixed bed; and recovering the target biomolecule.
12. The process according to any of the previous embodiments 2-11, wherein the target biomolecule is selected from the group consisting of: therapeutic proteins, antibodies, viruses, viral vaccines, viral vectors, viral genomes, proteins, virus-like particles (VLPs), microvesicles, exosomes, and polysaccharides.
13. The process according to embodiment 12, wherein the cells are selected from the group consisting of adherent cells, suspension cells and a combination of adherent and suspension cells.
14. The process according to embodiment 13, wherein the cells are adhered to or trapped in the fixed bed.
15. The process according to embodiment 14, wherein the target biomolecule is intracellular.
16. The process according to embodiment 15, wherein the target biomolecule is extracellular.
17. The process according to embodiment 16, wherein the cells are in a cell culture medium.
18. The process according to embodiment 17, wherein the cells are in a solution to detach cells from the fixed bed and the process further comprises a step of detaching the cells from the fixed bed.
19. The process according to embodiment 18, wherein the detaching solution comprises a combination of detergent, DNase, appropriate pH (different than isoelectric point) and appropriate conductivity.
20. The process according to embodiment 19, wherein the cells are in a lysis solution and the process further comprises a step of lysing the cells.
21. The process according to embodiment 20, wherein the lysis solution comprises a combination of a detergent, a DNase at an appropriate pH (different than isoelectric point) and appropriate conductivity.
22. The process according to embodiment 21, wherein moving the fixed bed comprises a motion selected from the group consisting of vibration, agitation, compaction, expansion and combinations of the foregoing.
23. The process according to embodiment 22, wherein moving the solution comprises a motion selected from the group consisting of vibration, agitation, compaction, expansion and combinations of the foregoing.
24. The process according to embodiment 23, wherein the detaching step is before the lysing step.
25. The process according to embodiment 24, further comprising adding a lysis solution and lysing the cells.
26. The process according to embodiment 25, wherein the bioreactor comprises lysed cells and the target biomolecule in solution.
27. The process according to embodiment 26, further comprising a step of adding a high conductivity solution, wherein the high conductivity solution comprises a conductivity between and including 150 mmol/l NaCl and 2 mol/l NaCl.
28. The process according to embodiment 27, wherein the high conductivity solution contacts the lysed cells and the target biomolecule in solution for a period of time from and including 5 and 360 minutes.
29. The process according to embodiment 28, further comprising an electrophoresis step.
30. The process according to embodiment 29, wherein at least a portion of the lysing step is conducted in a recirculation mode.
31. The process according to embodiment 30, wherein at least a portion of the high conductivity solution step is conducted in a recirculation mode.
32. The process according to embodiment 31, wherein the cells are detached from the fixed bed before the lysing step.
33. The process according to embodiment 32, wherein the cells are recovered from the fixed bed before the lysing step.
34. The process according to embodiment 33, wherein the cells are removed from the fixed bed before the lysing step.
35. A process for recovering a target biomolecule from cells in a fixed bed bioreactor, the process comprising the following steps, lysing the cells in the bioreactor by adding a lysis solution to the bioreactor;
increasing the conductivity of the lysis solution by adding a salt to the lysis solution; and recovering target biomolecule from the bioreactor.
36. The process according to embodiment 35, wherein the conductivity of the lysis solution is increased by adding NaCl to the lysis solution.
37. The process according to embodiment 36, wherein the concentration of NaCl in the lysis solution is between 1M and 2M.
38. A process for recovering a target biomolecule from cells in a fixed bed bioreactor, the process comprising the following steps,
lysing the cells in the bioreactor by adding a lysis solution to the bioreactor;
draining the lysis solution from the bioreactor;
adding a high conductivity solution to the bioreactor; and
recovering target biomolecule from the bioreactor.
39. The process according to any of the previous embodiments 2-38, wherein the target biomolecule is selected from the group consisting of: therapeutic proteins, antibodies, viruses, viral vaccines, viral vectors, viral genomes, proteins, virus-like particles (VLPs), microvesicles, exosomes, and polysaccharides.

### DESCRIPTION OF FIGURES

FIG. 1 shows a flow chart of the steps performed in a method for obtaining AAV2 according to an embodiment of the invention as described in example 1.

FIG. 2 shows a flow chart of the steps performed in a method for obtaining AAV2 according to an embodiment of the invention as described in example 2.

FIG. 3 shows a flow chart of the steps performed for harvest and recovery of target biomolecules from a fixed bed bioreactor under agitation at 1 cm/s according to an embodiment of the invention.

FIG. 4 shows a flow chart of the steps performed for harvest and recovery of AAV from a fixed bed bioreactor according to an embodiment of the invention.

FIG. 5 shows a flow chart of the steps performed for harvest and recovery of AAV from a fixed bed bioreactor according to an embodiment of the invention, while the bioreactor is operated in a recirculation mode.

FIG. 6A shows a general presentation of an electrophoresis-derived system.

FIG. 6B shows an electrode detailed structure according to an embodiment of the invention.

FIG. 7A shows migration of negatively charged biological molecules in an electrophoresis-derived system according to an embodiment of the invention. The negative particles are migrating from anode to cathode.

FIG. 7B shows circulation inversion during electrophoresis migration according to an embodiment of the invention, in order to collect easily the biological molecules and avoid their agglomeration on the cathode (+).

FIGS. 8-12 show a system for applying mechanical action to a bioreactor according to an embodiment of the invention.

FIG. 13 shows a bridge structure according to an embodiment of the invention comprising a strap guide for locating and guiding the placement of the straps during installation of the straps in the fixation system and a pushing pin adapted to limit displacement and/or breakage of the lid of the vessel or bioreactor.

FIG. 14 shows a ratchet preinstalled with a strap for adjusting the length of the strap in the fixation system according to an embodiment of the invention.

FIGS. 15 and 16 show a vibration table according to an embodiment of the invention.

### DETAILED DESCRIPTION OF THE INVENTION

The present invention concerns methods for obtaining viral vector and other target biomolecules from fixed bed bioreactors. In a second aspect, the present invention relates to a system comprising an agitation device and a fixation system for obtaining one or more target biomolecules from cells cultured in a fixed bed bioreactor.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

The expression “% by weight”, “weight percent”, “% wt” or “wt %”, here and throughout the description unless otherwise defined, refers to the relative weight of the respective component based on the overall weight of the formulation.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Fixed bed bioreactors are efficient tools to produce biological molecules such as recombinant proteins, monoclonal antibodies, viral vectors, viruses and cellular vesicles (such as exosomes) (also referred to as “target biomolecules”). Animal cells are used to produce such products (using constitutive expression systems, infection or transfection). The product(s) of interested can be released by the cells (such as secreted recombinant proteins, “secreted” viruses or lytic viruses) or can remain partially intracellular (e.g. intracellular proteins, viruses or some viral vectors).

Extracellular target biomolecules may be recovered in the supernatant of the bioreactor and the recirculation/perfusion loop(s), using one or more emptying, rinsing and concentrating steps to recover the target biomolecule of interest (referred to herein as target biomolecule).

Intracellular products stay in the cells and require lysing the cells at the end of the process using chemical/enzymatic solutions. After lysing the cells, the solution containing the cell debris (host cell protein, host cell DNA, cell membranes and cell organelles) and the target biomolecule is harvested by emptying and rinsing the bioreactor (as for extracellular products). The cell membranes are fragile and solutions to lyse the cells are known (hypo-hyper-osmotic shocks, acidic-basic-pH shifts, detergents or a combination of hose). However, most target biomolecules are easily damaged. Hence, there is a need for a solution that can lyse the cells but not damage the target biomolecule.

Some prior art processes detach the cells (with trypsin or cell detaching enzyme) to later lyse them outside of the bioreactor because it is not efficient to do so inside the bioreactor. Further, it is known that recovery of intracellular target biomolecules from cells in fixed bed bioreactors is difficult using classical methods. There is a need for efficient processes to recover intracellular biomolecules from fixed bed bioreactors.

Among those intracellular products, some target biomolecules are difficult to harvest because they are sensitive to pH, shear and detergent. In addition, many target biomolecules are known to stick or adhere to plastic materials, such as T-Flasks, cell factories and storage vessels. Further, some target biomolecules, including Adeno-Associated Virus, and the serotype 2 collectively referred to herein as AAV, may adhere to plastic bioreactor materials and plastic fixed bed materials, particularly hydrophilized plastic fixed bed and hydrophilized plastic structured fixed bed materials. Hence, there is a need for a solution that will help in the recovery of a target biomolecule from a fixed bed bioreactor by reducing the target biomolecule's interaction with one or more plastic parts including but not limited to the bioreactor and the fixed bed. Further, it is believed that some target biomolecules, particularly intracellular target biomolecules including but not limited to AAV may, after a lysing step, remain mechanically trapped in or adhere to the fixed bed and cell debris (including organelles such as nuclei) making recovery of the target biomolecule difficult. Hence, there is a need for a solution that will help in the recovery of a target biomolecule from a fixed bed bioreactor by reducing the target biomolecule's interaction with the fixed bed and cell debris. The addition of surfactants (such as Pluronic F68—usually at 0.1-0.01%) to a AAV suspension cell culture harvest is known in the art to prevent “losing” viruses that remain on bioreactor, tubing or storage vessel walls.

The inventors discovered that when they attempted to recover AAV from cells in a bioreactor with a structured fixed bed after a lysis step in the bioreactor using solutions and processes known in the art for suspension cell technologies, a large percentage of AAV was not recovered because it was trapped in or adhered to the fixed bed or to cellular debris (such as cellular host cell proteins/DNA) which was trapped in or adhered to the fixed bed.

The inventors also discovered that when the structured fixed bed material comprised hydrophilized PET, the majority of the unrecovered target biomolecule (e.g., AAV) interacted/aggregated with and/or was trapped with cell debris, adsorbed protein, adsorbed DNA and extracellular cell matrix that remained on the fixed bed material after chemical (detergent) lysis of the cells.

Disposable fixed bed bioreactors (examples include Pall's iCELLis, Univercells Technologies' scale-X and Corning's Ascent) are composed of plastic material on which the biomolecules (e.g., AAV) could stick. As they have substantial hydrophilized surface for the cells, the “stickiness” of AAV or other target biomolecules is an important concern, making recovery of target biomolecules more complex than from suspension cell bioreactors.

The inventors tested a scale-X bioreactor with a classical solution of 0.1 and 1% Triton and DNase (benzonase) and observed that some viruses remain after the harvest (by fluorescent microscopy and ex-situ harvest after a freeze/thaw cycle post in-situ harvest).

Information derived from tests performed on AAV2 harvest from a scale-X bioreactor showed:
A ‘Classical solution’ based on detergents is sometimes not sufficient (0.1 and 1% triton X-100) to have good recovery yield (viruses remain after the in-situ lysis and rinse—proof performed by fluorescent microscopy and ex-situ harvest performed post the ex-situ lysis).
‘Classical solutions’ are sufficient to lyse the cells (proof: turbidity of the solution) but not to recover the viruses.
   Addition of benzonase (DNase) to degrade the DNA to limit the stickiness of the viruses is sometimes not sufficient.

Concerning the lysis solution, Triton X-100 and Tween (tween 20 and tween 80) detergents are known to be efficient to lyse cells and recover AAV from suspension cells (with concentration from 0.1 to 0.5%). DNase (such as benzonase) could be added to cleave the free DNA and prevent the AAV to stick to DNA. However, recovery of the target biomolecule is still problematic as described hereinabove.

The inventors surprisingly found that combining a chemical lysis step (using a lysis solution) with mechanical action helps to recover target biomolecules from the bioreactor. In one embodiment the present invention comprises novel combinations of solutions to improve the yield of target biomolecules, preferably intracellular biomolecules from cells in a cell culture or cell harvest solution.

As such, in a first aspect, the invention relates to a method for obtaining one or more target biomolecules from cells cultured in a fixed bed bioreactor, said method comprises:
the addition of a lysis solution to the bioreactor, thereby lysing the cells in the bioreactor, and prior to, simultaneous with, and/or after lysing said cells, applying one or more mechanical actions to said bioreactor, after which the one or more target biomolecules are recovered from said bioreactor.

Although the current disclosure is presented primarily in the context of recovering AAV from cells, the inventions described herein shall not be limited to recovery of AAV but such inventions may be used to recover other target biomolecules from adherent or suspension cells, wherein such target biomolecules may comprise one or more of viruses, non-lytic viruses, viruses with limited cytopathic effect, non-secreted or partially-secreted recombinant protein, non-secreted or partially-secreted antibodies, intracellular or partially intra-cellular viral vectors. The cells can be selected from the group consisting of adherent cells, suspension cells and a combination of adherent and suspension cells. All types of cells can be used, examples include mammalian cells, such as stem cells (for instance hematopoietic stem cells, skeletal muscle stem cells, mesenchymal stem cells), immune cells (for instance lymphocytes, dendritic cells) and pancreatic islet cells. Prokaryotic cells (such as bacterial cells) or viral cells can also be used.

In an embodiment, said target biomolecules are AAV. As such, in an embodiment, the invention relates to a method for obtaining AAV from cells cultured in a fixed bed bioreactor, said method comprises:
the addition of a lysis solution to the bioreactor, thereby lysing the cells in the bioreactor, and prior to, simultaneous with, and/or after lysing said cells, applying one or more mechanical actions to said bioreactor, after which AAV molecules are recovered from said bioreactor.

In one embodiment the present invention comprises one or more of the following steps to recover target biomolecules, preferably AAV viruses from a bioreactor, preferably a fixed bed bioreactor.

As described above, adsorption of the biomolecules to the plastic vessel walls occurs. In an embodiment, the method of the invention hence comprises using surfactant to limit the adsorption of biomolecules such as AAV to plastic vessel walls. In an embodiment, 0.1% of surfactant is used (more than for usual processes). In an embodiment, 0.1 to 0.01% Pluronic F68 is used. In an embodiment, the method of the invention further comprises using surfactant to limit the adsorption of biomolecules such as AAV to plastic vessel walls (as for suspension processes—e.g., 0.1 to 0.01% Pluronic F68). Pluronic™ F-68 is a non-ionic surfactant generally used to control shear forces, prevent foam production in stirred culture and reduce cells attachment to hydrophilic surfaces. It is also known to be added to prevent biomolecules such as AAV to stick to plastic of pipets and plastic vessels during storage. Usual concentration advice for AAV is between 0.01 and 0.2%.

In an embodiment, the method of the invention further comprises performing several cycles with several rinses to recover target biomolecules, preferably AAV viruses from a bioreactor, preferably a fixed bed bioreactor.

In an embodiment, the bioreactor is first drained before a lysis solution is added to the bioreactor. In a further embodiment, 0.1% of Pluronic™ F-68 (v/v) is added to the culture supernatant between 30 minutes and 1 hour before draining the bioreactor. There is already Pluronic™ F-68 at 0.1% (v/v) in some culture media. Even if there is Pluronic™ F-68 in the culture media, it could be useful to add Pluronic™ F-68 until reaching 0.2% (v/v). As such, in an embodiment, Pluronic™ F-68 is added until reaching a concentration of 0.2% (v/v) in the culture supernatant.

In an embodiment, the bioreactor is first drained and rinsed before a lysis solution is added to the bioreactor. Such a neutral rinsing solution is used to eliminate the residual culture medium (components that could interfere with the lysis step) and to pursue the collect of the biomolecules (such as rAAV2) extracellular fraction. In an embodiment, once the bioreactor is empty, it is completely filled with the rinsing solution and emptied afterwards, thereby performing a first rinsing step. The inventors observed that filling/emptying of the bioreactor has a positive effect on the harvest of biomolecules such as rAAV. As such, in an embodiment, more than one rinsing step is performed.

In an embodiment, a rinsing buffer or rinsing solution formulation is used during said rinsing steps.

In a further embodiment, said rinsing buffer comprises a surfactant, such as Pluronic. In a further embodiment, said rinsing buffer comprises 0.1% (v/v) Pluronic. In an embodiment, said rinsing buffer comprises PBS-MK buffer (PBS-Magnesium Potassium buffer) and Pluronic™ F-68. In an embodiment, said rinsing buffer comprises PBS, 2.5 mM KCl, 1 mM MgCl₂, 0.1% (v/v) Pluronic, pH 7.

Cell lysis forms the central step of the method of the invention, because this is necessary to release the intracellular target biomolecules (for instance the majority of rAAV2 is intracellular). The contact time between the cells and the lysis solution, as well as the temperature at which the lysis is carried out, influences the efficiency of the lysis.

In an embodiment, the lysing step comprises adding a lysis solution to the bioreactor and contacting the cells with the lysis solution for a period of time sufficient to lyse at least a portion of the cells. In an embodiment, the lysis solution contacts the cells for 30 minutes to 6 hours. In an embodiment, the lysis solution contacts the cells for 1 to 2 hours. In an embodiment, the method of the invention comprises having sufficient contact time between the cells and the lysis solution to recover target biomolecules, preferably AAV viruses from a bioreactor, preferably a fixed bed bioreactor. During this contact time one or more mechanical actions should be applied to the bioreactor. For instance the bioreactor could be agitated.

In a preferred embodiment, the lysis step is performed for 2 hours (could depend on the processes) at 37° C. while maintaining 0.5-1 cm/s agitation (moving a liquid level relative to the fixed bed structure at a speed of 0.5 to 1 cm/s. The stirring can regularly be switched on/off to disrupt the flow. Back and forth cycles (emptying/filling steps) could be also performed as wash-out effect on the fixed bed helps to recover the target biomolecules, such as the viruses. Alternatively, lysis steps could be done two times for one (or two) hours each. The lysis step could take, based on the scientific literature, between 30 min to several hours (up to 6 hours) (e.g., 4 hours) with regular sampling of the raw lysate (e.g., after 1, 2, 3, 4 hours). These samples can then be analyzed to quantify the viral titer. Virus should be stable for several hours in these lysis solutions.

In an embodiment, the lysis solution comprises a detergent reagent. Detergent reagents for lysing cells are known in the art and a particular detergent may be selected based on the cell line used, the target biomolecule and the type of bioreactor. In an embodiment, said lysis solution comprises at least a detergent, such as Triton X-100, Tween 20 or Tween 80.

Tween20 or Triton X-100 is used because detergents disrupt the cell membrane. Use of detergents is concentration and time dependent, with reported concentrations of Triton X-100 between 0.1% and 0.5% and of Tween 20 between 0.1 and 1% (v/v). Zwittergent 3-14 (Calbiochem) seems also promising. Since European REACH regulation tends to ban Triton and its derivates, the use of Tween20 or Zwittergent is favored. Triton is known to be more efficient than Tween. Higher concentrations of tween are recommended (Triton or Zwittergent concentration for sufficient virus release are lower compared to Tween 20. This is linked to an intrinsic property of the detergent, the critical micellar concentration).

In an embodiment, the lysis solution may comprise a detergent reagent or surfactant selected from the group consisting of Triton X-100, Tween-20, Tween-80, Zwittergent and combinations thereof. In an embodiment, the concentration of the detergent reagent in the lysis solution is from 0.05 to 1.5% v/v. In a preferred embodiment, the concentration of the detergent in the lysis solution is between 0.1 to 1.0% v/v.

In one embodiment, the cell line comprises HEK293T cells. In an embodiment, the bioreactor is a structured fixed bed bioreactor and in a further embodiment, the target biomolecule is intracellular. In an embodiment, the target biomolecule may comprise an intracellular protein, a virus or a viral vector. In a preferred embodiment, the target biomolecule is AAV.

In an embodiment, said lysis solution further comprises a DNase and/or a surfactant.

In an embodiment, the method of the invention comprises using DNase to degrade the free DNA and prevent that biomolecules such as AAV stick to DNA adsorbed on the fixed bed fibers. In a further embodiment, DNase can be used for suspension processes, wherein e.g. 10-50 Units of Benzonase/ml can be used at 37° C. for 1 hour in 1-2 mM MgCl₂ in classical phosphate buffers.

In an embodiment, nuclease treatment is performed during the lysis step. DNase is added to:
1. Digest the host cellular nuclear material during extraction of the biomolecules such as rAAV
2. Avoid the formation of nucleic material—biomolecules such as AAV complexes
3. Reduce the viscosity of the lysate to facilitate subsequent filtration and chromatography steps.

To limit the complexes and viscosity induced by genomic DNA, it is preferable to add the enzyme concomitantly with cell breakage. However, this step may not be compatible with the optimal conditions for enzyme activity (pH & salinity such as NaCl). In an embodiment, Benzonase (Millipore Sigma) is used as a DNase. pH 8 doesn't alter the enzyme efficacy of Benzonase, since it is active between pH 7-9. However, the salt concentration is too high (1M NaCl; need to be lowered at 100-150 mM to keep an effective activity). In an embodiment, salt activated nuclease (SAN-HQ; ArcticZymes) can be used. Its activity at 500 mM NaCl is reported.

In an embodiment, 20-50 U/mL of Benzonase are added during the lysis step for a minimum incubation time of 30-60 min. Also, it may be necessary to add 1-2 mM MgCl₂ for the enzyme activity. Note that higher concentration of Benzonase could also be used.

As described above, in an embodiment, the lysing step may further comprise addition of an enzyme reagent to the lysis solution. In an embodiment, the enzyme reagent is selected from the group consisting of salt activated nuclease endonuclease and DNase. In another embodiment, the enzyme is a DNase enzyme and more potentially DNase, and more perhaps Benzonase. Accordingly, the lysis solution further comprises an enzyme reagent. Addition of an enzyme reagent is believed to hydrolyze and cleave free DNA and reduce the clumping or viscosity caused by the DNA content during cell lysis. The DNase may also assist in preventing sticking of the target biomolecule to DNA. The enzyme or DNase may be added to the lysis solution concomitantly with the detergent reagent or after addition of the detergent reagent. In an embodiment, the DNase is added to the lysis solution concomitantly with the detergent reagent. In an embodiment after addition of the detergent reagent (lysing agent). In an embodiment, the pH and conductivity are adjusted to optimize the enzyme activity. In an embodiment, the pH of the lysis solution is between 7 and 9 and the conductivity of the solution is between 50 and 150 mM. In an embodiment, the lysis solution comprising DNase, perhaps Benzonase incubates for 20 to 90 minutes. The lysis solution comprising DNase may incubate for 30-60 minutes.

In an embodiment, the method of the invention further comprises using DNase to degrade the free DNA and prevent that AAV stick to DNA adsorbed on the fixed bed fibers (as for suspension processes—e.g., 10-50 U Benzo/ml, 37° C., 1 hour, 1-2 mM MgCl₂ in classical phosphate buffers) to recover target biomolecules, preferably AAV viruses from a bioreactor, preferably a fixed bed bioreactor.

In an embodiment, the lysing step may comprise adding a DNase reagent to the bioreactor and contacting the cells with the DNase reagent for a period of time sufficient to hydrolyze at least a portion of the cells.

In an embodiment, the lysis solution comprising DNase, such as Benzonase, further comprises a salt to regulate the conductivity of the lysis solution. Preferably the conductivity of the lysis solution is optimal for the enzyme. In an embodiment, the salt is selected from the group consisting of NaCl, MgCl₂ and combinations thereof. In an embodiment, the lysis solution comprising benzonase, further comprises Mg cation in a concentration less than 150 mM, preferably between 100 and 150 mM. In an embodiment, the lysis solution comprising detergent, enzyme, and between 100 and 150 mM of salt, preferably Mg cation or NaCl and having a pH between 7 and 9, incubates for from 30 minutes to 2 hours.

In a preferred embodiment, the lysis solution comprises a DNase, a detergent or a surfactant or any combination of any of the foregoing, at the appropriate pH and conductivity.

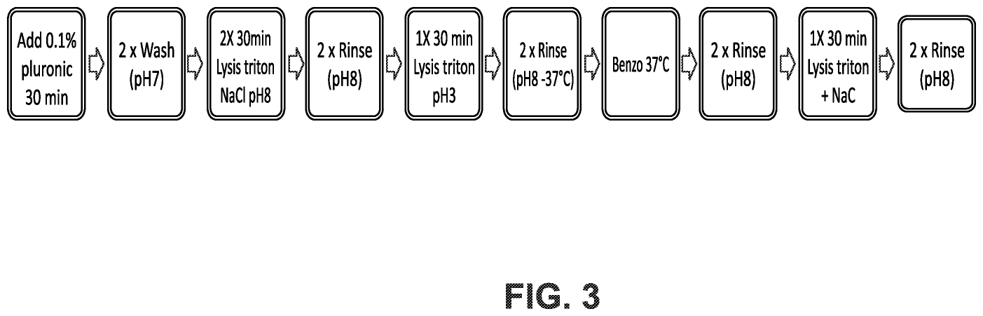

In an embodiment, the target biomolecule cell culture harvest may comprise up to three lysing steps as shown in FIG. 3. In an embodiment, the target biomolecule cell culture harvest may comprise a separate step for adding the enzyme (Benzo) as further shown in FIG. 3.

In an embodiment, the method of the invention further comprises using high conductivity solutions (0.5 to 1 M NaCl) to limit interactions between the biomolecules such as AAV and the adsorbed biological material remaining on fibers. The use of high conductivity solutions is especially useful when the target biomolecule is AAV.

In an embodiment, the method further comprises using a solution comprising 0.5 to 1 M NaCl during the lysing step and/or the washing step. In a further embodiment, said solution is obtained by increasing the ionic strength of the lysis solution during the lysing step.

NaCl is added because a sufficient ionic strength must be maintained to avoid rAAV2 aggregation and binding to other cellular components released during lysis. In an embodiment, a high salt concentration is used during the lysis step. We suggest that it also reduces the surface interactions between biomolecules such as rAAV2 and cells or cell debris that could remain adsorbed on bioreactor surfaces.

In an embodiment, the method of the invention further comprises using high conductivity solutions (0.5 to 1.0 M NaCl) to limit interactions between the target biomolecules such as AAV and the adsorbed biological material remaining on fibers.

In an embodiment, the invention relates to a lysis solution comprising 2.0 M NaCl. In an embodiment, the invention relates to a lysis solution comprising 0.5 to 1.0 M NaCl. In an embodiment, the invention relates to a lysis solution comprising 0.5 M NaCl. In an embodiment, the invention relates to a lysis solution comprising 0.6 M NaCl. In an embodiment, the invention relates to a lysis solution comprising 0.7 M NaCl. In an embodiment, the invention relates to a lysis solution comprising 0.8 M NaCl. In an embodiment, the invention relates to a lysis solution comprising 0.9 M NaCl. In an embodiment, the invention relates to a lysis solution comprising 1.0 M NaCl.

In an embodiment, the invention relates to a bioreactor comprising a fixed bed and a lysis solution comprising between 0.5 to 1.0 M NaCl.

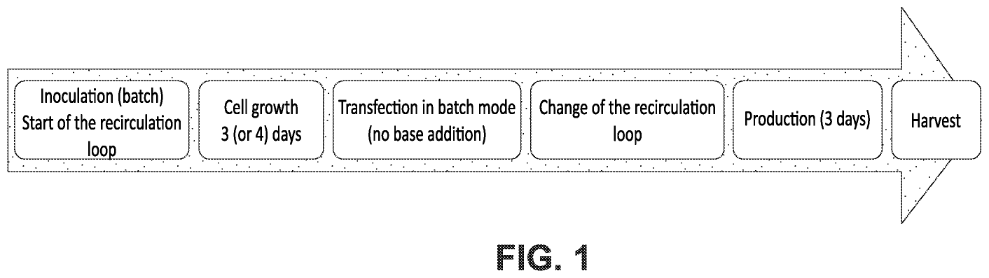

The present disclosure provides a process and chemistry for increasing the yield of a target biomolecule recovered from a cell culture harvest. More particularly, the disclosure provides a process for recovering a target biomolecule from a cell culture harvest in a bioreactor comprising a fixed bed, and a cell culture solution, said process comprising lysing cells in the bioreactor in a first step (step 2 in FIG. 1 and step 3 in FIG. 3) and increasing the conductivity of the cell culture solution in a second step. Increasing the conductivity of the cell culture solution, may help to neutralize the charge of the target biomolecule.

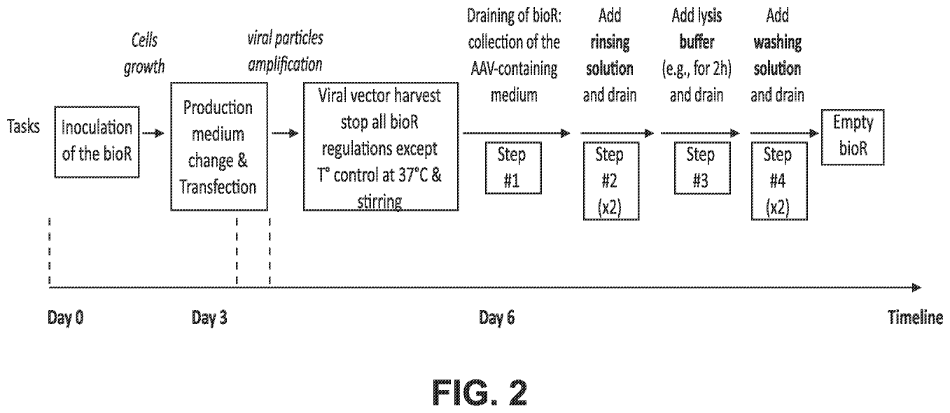

In an embodiment, after incubation with the lysis solution, the conductivity of the lysing solution is increased in a second step by adding additional salt to the lysis solution. Raising the conductivity of the lysis solution serves to increase the solubility of the target biomolecule that has aggregated with cellular debris during the lysis step and may further decrease the viscosity of the lysis solution. Increasing the solubility of the target biomolecule increases the concentration of free target biomolecule available for recovery in the lysis solution. In an embodiment in the second step of the lysis step, the conductivity of the lysis solution is increased to greater than 150 mM. In an embodiment, NaCl is added to the lysis solution after incubation with the lysis solution. In an embodiment the NaCl concentration is adjusted to up to 1.0 M after incubation with the lysis solution. In an embodiment the NaCl concentration is adjusted to up to 2.0 M after incubation with the lysis solution. In embodiments where there are multiple lysing steps, the conductivity of the lysis solution is increased after incubation with the final lysis solution. In an embodiment, target biomolecule is recovered from the high conductivity lysis solution. In an embodiment, target biomolecule is recovered from the high conductivity lysis solution in a draining step (see Step #3 in FIG. 2). In an embodiment, target biomolecule is recovered from the high conductivity lysis solution in a recirculation loop. In an embodiment, where there is more than one lysing step, target biomolecule is recovered from the lysis solution in one or more draining steps. Another possibility is to conduct a separate additional step with enzyme/endonuclease/DNase/Benzonase right after lysis to limit interference between cells lysis and endonuclease activity. Since the lysis step would be conducted with a high ionic strength this additional step with endonuclease should be done with a salt activated nuclease (SAN). Salt active nuclease is a general, unspecific endonuclease that cleaves double-stranded and single-stranded DNA, and RNA. SAN is active at above neutral pH. Unlike other nucleases, however, it has optimum activity at high concentrations of salt at high pH. These qualities make SAN ideal for removal of DNA from cell extracts and protein samples.

In an embodiment, the lysing step may be implemented in different steps. In an embodiment, addition of detergent and enzyme may be in separate steps. In an embodiment, the bioreactor is drained between each step and target biomolecule is recovered during each drain step. In an embodiment, detergent and enzyme and high conductivity solution are implemented in separate steps. In an embodiment, the bioreactor is drained between each step and target biomolecule is recovered during each drain step. In an embodiment, detergent and enzyme are added in one step and high conductivity solution is added in a separate step. In an embodiment, the bioreactor is drained between each step and target biomolecule is recovered during each drain step.

In an embodiment, the bioreactor is agitated during the lysis step. Systems and processes for agitating the bioreactor are described hereinbelow. In an embodiment, any one or more lysing steps may be implemented in a recirculation mode. In an embodiment, recovery of target biomolecule may occur during any step and/or from any one or more solutions described hereinabove or hereinbelow, including where a step is operated in a recirculation mode. In one or more embodiments recovery of target biomolecule includes collection of the target biomolecule. In an embodiment, target biomolecule is recovered and collected from cell culture harvest supernatant before the lysis step (Step #1 in FIG. 2). In an embodiment, the bioreactor is drained, and target biomolecule is recovered and collected from the cell culture harvest supernatant before the lysis step (Step #1 in FIG. 2). In an embodiment, a surfactant, preferably poloxamer 188 (also known as Pluronic) is added to the cell culture harvest before or after draining the cell culture harvest supernatant and before the lysis step (step 1 in FIG. 3). Surfactant, preferably, poloxamer 188 (also known as Pluronic F68) is used to reduce or control foaming and/or clumping and/or to control shear forces and/or to reduce cell attachment to hydrophilic surfaces in the cell culture harvest, and particularly in the supernatant. In an embodiment, surfactant, preferably polaxamer 188 is added to the cell culture harvest supernatant for between 15 and 45 minutes before the lysis step and the concentration of polaxamer 188 in the cell culture harvest supernatant before lysis is between 0.05% w/v and 5.0% w/v. In an embodiment, the cell culture harvest supernatant comprising the polaxamer is drained from the bioreactor and target biomolecule is recovered and collected from the cell culture harvest supernatant after the addition of polaxamer and before the lysis step (Step #1 in FIG. 2 and Step 1 in FIG. 3). In an embodiment, a rinse step comprising addition of a rinse solution to the bioreactor after draining of the cell culture harvest supernatant from the bioreactor (see step 2 in FIG. 2 and step 2 in FIG. 3). The rinse solution may eliminate residual culture medium that could interfere with the lysis step. In an embodiment, the rinse solution comprises a buffer. In an embodiment the buffer comprises PBS. In an embodiment the pH of the wash solution is between 6 and 8. Preferably the pH of the rinse solution is 7. In an embodiment, the wash solution further comprises a surfactant. In an embodiment, the surfactant is polaxamer. In an embodiment the rinse solution is drained from the bioreactor. In an embodiment, the rinse step is repeated from 2 to 4 times. Preferably the rinse step is repeated twice.

In an embodiment, the rinse solution comprises:
PBS
2.5 mM KCl
1 mM MgCl2
0.1% (v/v) Pluronic
pH 7

In an embodiment, wash steps are carried out after each lysis step (see Step #4 in FIG. 2 and intermittent rinse steps in FIG. 3). Wash steps recover target biomolecule remaining in the bioreactor and the fixed bed of the bioreactor after the lysis step. In an embodiment, the wash steps recover target biomolecule remaining in one or more of the fixed bed (fibers, cells debris, extracellular matrix) and in interaction with the bioreactor walls or interior components. In an embodiment, a washing solution is added to the bioreactor. In an embodiment, the washing solution contacts the interior of the bioreactor and the fixed bed for a period of time sufficient to recover target biomolecule into the washing solution from the bioreactor and the fixed bed. In an embodiment, the rinse solution is drained from the bioreactor and target biomolecule is recovered from the washing solution. In an embodiment, the washing and draining steps are repeated. In an embodiment, the washing and draining steps are repeated from 2 to 4 times. In an embodiment, the washing and draining steps are repeated twice.

In an embodiment the washing solution comprises:
PBS
1.0 to 3.0 mM KCl
0.5 to 2.5 mM MgCl₂
0.01 to 0.2% (v/v) Pluronic
5 to 15 mM Tris-pH 8

In another embodiment, the washing solution comprises:
PBS
2.5 mM KCl
1 mM MgCl2
0.1% (v/v) Pluronic
10 mM Tris-pH 8

In an embodiment, the bioreactor may be agitated during any one or more of the cell culture harvest steps described hereinabove or hereinbelow using any of the techniques or processes described hereinbelow. In an embodiment, agitation increases the amount of target biomolecule recovered from the bioreactor.

In one embodiment, the inventors observed that pH below or above the isoelectric point of the AAVs (between 5 to 6 depending on the serotypes) have better recovery yield than neutral pH (ex-situ harvest at pH 3.0 and 8.0). The inventors have also shown that high conductivity allows to have better recovery yield.

As described above, it is believed that the AAV stick to the adsorbed biological material on the fibers of the fixed bed. The inventors have shown that not neutral pH (low: 3.0-4.0 and high: 8.0-9.0) and a high conductivity solution (1M NaCl) help to recover the virus, indicating that electrostatic interactions are key elements in the adhesion of the AAV to the adsorbed biological material on the fibers of the fixed bed. The inventors did some tests to prove this and observed that pH below or above the isoelectric point of the AAVs (between 5 to 6 depending on the serotypes) have better recovery yield than neutral pH (tested by performing ex situ harvest at pH 3.0 and 8.0). The inventors have also shown that high conductivity allows to have better recovery yield (tested by performing ex-situ harvest at 0.5 and 1 M NaCl). Those solutions are quite similar to solutions to elute viruses from IEX (ion exchange chromatography) and CEX (cation exchange chromatography) and are suitable for the AAV stability. Hence, in one embodiment the present invention is to combine cell lysis chemical solutions (detergents) to solutions that are commonly used to elute viruses from IEX and CEX chromatography systems to recover the viruses from the fixed bed.

In an embodiment, the method of the invention comprises using a pH below the isoelectric point (pH 3 to 5) or above the isoelectric point (pH 8 to 9).

In an embodiment, the method of the invention comprises using a pH below the isoelectric point or above the isoelectric point of the target biomolecule. By using a pH below the isoelectric point or above the isoelectric point of the target biomolecule the interaction between the target biomolecules and the hydrophilized fixed bed material is prevented.

Note that as viruses are not well stable at pH 3, the solution should be quenched and buffered after the lysis.

As such, in a further embodiment, after the lysis step, the solution is quenched and buffered. In an embodiment, an inactivation solution is added after the lysis step to quench or inhibit the lysis solution—in order to improve the stability of the biomolecules after the harvest.

A high ionic strength (NaCl>150 mM) is important to avoid viral vector aggregation, but these monovalent salts concentrations largely reduce Benzonase activity (e.g., +/−70% loss of relative activity at 150 mM NaCl). To ensure an efficient harvest, the following tradeoff can be used:

In an embodiment, first, the lysis step begins without adding NaCl (N.B.: 137 mM provided by PBS buffer) to maintain an effective activity of the Benzonase (producer's brochure). As Mg²⁺ is a cofactor of Benzonase, its concentration must be accurate. Thus, in an embodiment, 2 mM MgCl₂ is added, this concentration being optimal for the enzyme activity. Under these salinity conditions, the Benzonase retains an effective activity and vector aggregation should be limited. In a next step, after 1 (or 2) hours of incubation, the ionic strength of the lysis buffer can be increased by adjusting the NaCl concentration up to 1M. Under these conditions, Benzonase is inhibited but the risk of viral vector aggregation is reduced and/or the aggregates of virus particles solubilize (if the process is reversible). At such salinity, the virus interaction with plastic material should be reduced.

In another embodiment, a separate additional step with endonuclease/Benzonase is conducted right after lysis to limit interference between cells lysis and endonuclease activity. Since the lysis step would be conducted with a high ionic strength this additional step with endonuclease should be done with a salt activated nuclease.

In an embodiment, the lysis buffer formulation is as follows:
1% (v/v) Tween20 or Triton X-100
1M NaCl
10 mM Tris-pH 8
0.1% Pluronic (v/v)
20-50 U/mL endonuclease (Dnase) & 1-2 mM MgCl2

A 10 mM Tris-pH 8 buffer is used because it is observed that an alkaline solution plus appropriate amounts of detergents such as Triton X-100 or Tween20 are sufficient to lyse cells packaged with biomolecules (for instance rAAV packaged cells) and release said biomolecules (for instance viral particles) from the cells. Depending on the process, chemical cell lysis protocols report pH values generally between 8-9. An acidic pH (3-4) seems also promising to harvest biomolecules (for instance viral particles), but it might have an impact on the integrity of the (viral) proteins.

The function of 0.1% Pluronic (v/v) is similar to its function in the rinsing solution. Pluronic™ F-68 is a non-ionic surfactant generally used to control shear forces, prevent foam production in stirred culture and reduce cells attachment to hydrophilic surfaces. It is also known to be added to prevent AAV to stick to plastic of pipets and plastic vessels during storage.

In one embodiment, the invention comprises a method for recovering a target biomolecule, preferably AAV, more preferably intracellular AAV by combination of detergent, Pluronic (prevent AAV adsorption), high conductivity, pH and benzonase (cleave the host cell DNA).

As described above, in a preferred embodiment, after said lysing of said cells, one or more washing steps are performed by filling and/or draining the bioreactor with a washing solution.

Washing steps are conducted to remove the biomolecules such as rAAV2 blocked in the fixed bed (fibers, cells debris, extracellular matrix) and/or in interaction with the bioreactor's walls. Once the bioreactor is empty, it is filled with the washing solution and then emptied in order to perform a washing step. As discussed above, filling/emptying the bioreactor has a positive effect on the harvest of biomolecules such as rAAV. As such, in a preferred embodiment, the washing step is performed more than once, for instance two times. In an embodiment, the washing step is performed after 2 hours of cell lysis.

In an embodiment, the washing solution formulation is as follows:
PBS
2.5 mM KCl
1 mM MgCl₂
0.1% (v/v) Pluronic
10 mM Tris-pH 8

In a preferred embodiment, compared to the rinsing solution, the pH of the washing solution is more alkaline.

When cells growing on a surface are lysed, mechanical actions or motions should be applied to recover the cell debris and the product of interests that could remain stuck to the surface. In some cases, only gentle agitation is sufficient. Sometimes, just the movement of the liquid is sufficient to recover the product of interest. But sometimes stronger mechanical actions are required. Recovering biomolecules such as AAV from fixed bed bioreactors is more complex than from suspension cells because said biomolecules such as AAV could stick to the surfaces of the bioreactors (surface available to grow the cells).

As such, the invention described hereinabove, includes mechanical actions to recover and/or improve recovery of cells (adherent or suspension cells) from the fixed bed and/or the bioreactor. The inventors performed tests on AAV2 harvest from a fixed bed bioreactor, that showed that the AAV do not stick to ‘fresh’ hydrophilized PET/PP material (non-colonized fixed bed fibers), indicating that the AAV do not stick to the fixed bed material (PET/PP) itself, but remained trapped by cell debris or other biomolecules (e.g. cell membranes, DNA, proteins etc.) that remain blocked inside the fixed bed structure after chemical (detergent) lysis.

AAV harvest tests performed (ex-situ) at rest, under light agitation and strong mechanical actions (vortex) indicated that mechanical actions play a role in the AAV harvest yield.

The current invention relates to a method for obtaining one or more target biomolecules from cells cultured in a fixed bed bioreactor, said method comprises:
the addition of a lysis solution to the bioreactor, thereby lysing the cells in the bioreactor, and prior to, simultaneous with, and/or after lysing said cells, applying one or more mechanical actions to said bioreactor, after which the one or more target biomolecules are recovered from said bioreactor.

The invention comprises a harvesting process for recovering a target biomolecule from a cell culture harvest in a bioreactor comprising a fixed bed, wherein the process comprises applying mechanical action to the bioreactor, the fixed bed or a combination thereof for a period of time sufficient to improve the recovery of the target biomolecule.

In an embodiment, said mechanical action can be selected from moving the fixed bed bioreactor or moving a solution in said fixed bed bioreactor, thereby moving a liquid level relative to the fixed bed structure. Said liquid level can be moved at a certain speed, thereby expressing the distance moved by the liquid level in relation to the fixed bed structure in a certain time frame (for instance expressed in cm/s).

In a further embodiment, moving the bioreactor comprises a motion selected from the group consisting of vibration, agitation, compaction, expansion, rocking, applying ultrasound waves and combinations of the foregoing.

In an embodiment, moving the fixed bed bioreactor comprises moving the fixed bed inside said bioreactor. In a further embodiment, moving the fixed bed comprises a motion selected from the group consisting of vibration, agitation, compaction, expansion and combinations of the foregoing.

In a further embodiment, moving a solution comprises a motion selected from the group consisting of vibration, agitation, compaction, expansion, rocking, applying ultrasound waves, at least partially draining the bioreactor of liquid, adding liquid to the bioreactor and combinations of the foregoing. Said solution can be any type of suitable solution, such as a lysis solution, a washing solution or an inactivation solution. The solution (for instance the lysis solution) can be moved internally by using internal circulation within the bioreactor (such as via an agitator), or by using external recirculation (circulation or perfusion). In a further embodiment, said solution can be moved internally by using internal circulation within the bioreactor by means of an impeller

In a preferred embodiment, the method includes agitating the bioreactor and moving a solution in said fixed bed bioreactor, thereby moving a liquid level relative to the fixed bed structure. In a further preferred embodiment, the agitating and moving steps are done simultaneously. By moving a liquid level relative to the fixed bed structure, simultaneously with agitating the bioreactor (for instance by means of vibrations) the energy of the agitation can be transferred to the Air Liquid interface (ALI) of the fixed bed structure. The moving step may comprise at least partially draining the bioreactor of liquid, such as by moving the liquid level from adjacent a top of the fixed bed structure to adjacent a bottom of the fixed bed. The moving step may comprise adding liquid to the bioreactor, such as for example by adding a cell lysis solution to the bioreactor. The moving step may comprise moving the fixed bed relative to the bioreactor to move a location of the liquid level. The liquid level may be located above the fixed bed structure prior to the moving step, which may involve raising and lowering the liquid level (such as from the top of the fixed bed structure to the bottom of the fixed bed structure, for example), a plurality of times (but it could also be only one time). The agitating step may comprise vibrating the bioreactor.

The method may include adjusting a position of a liquid level in the fixed bed bioreactor while vibrating the bioreactor. The adjusting step may comprise filling and flushing the bioreactor with liquid, including by repeatedly filling and flushing the bioreactor with liquid.

Still further, the method may comprise tilting the bioreactor and/or compacting a fixed bed in the bioreactor. The adjusting step may comprise moving the fixed bed relative to the bioreactor.

The method may include vibrating the bioreactor and tilting and draining the bioreactor. The vibrating, tilting, and draining steps may be performed concurrently.

In a preferred embodiment, the biomolecule is an intracellular biomolecule. In a more preferred embodiment, the target biomolecule is an intracellular protein, virus or viral vector. The mechanical action may be applied constantly or intermittently and during one or more target biomolecule harvesting steps; for a period of time, sufficient to improve the recovery of the target biomolecule. In an embodiment, the mechanical action is vibration. In another embodiment the mechanical action is rotation. In a still further embodiment, the mechanical action is a combination of different actions.

In one embodiment, the mechanical action is applied after a cell culture. In another embodiment, the mechanical action is applied during a cell harvesting process. In one embodiment the cell culture harvest comprises animal cells. In a further embodiment the cell culture harvest comprises HEK293T cells. In another embodiment, the mechanical action is applied during one or more target biomolecule harvesting steps of a target biomolecule harvest process. In another embodiment, the mechanical action is applied during one or more target biomolecule harvesting steps comprising one or more of, before medium is drained from the bioreactor, while medium is drained from the bioreactor, after medium is drained from the bioreactor, during addition of a rinsing solution, during a rinsing step, during draining of a rinsing solution, before addition of a lysis solution, during addition of a lysis solution, during a lysing step, during draining of lysis solution, after draining of a lysis solution, before addition of a washing solution, during addition of a washing solution, during a washing step, during draining of a washing solution and after draining of a washing solution.

In one embodiment, the mechanical action applied during any one or more steps in the target biomolecule (including cellular vesicles) harvesting process including the steps listed hereinabove and the steps listed hereinbelow, may comprise any one or more processes or devices known to one skilled in the art used to apply mechanical action to a bioreactor, a fixed bed or a combination thereof. In one embodiment, the mechanical action applied during any one or more steps in the target biomolecule harvesting process including the steps listed hereinabove and the steps listed hereinbelow, may comprise any one or more processes or devices disclosed in PCT publication WO2022/254039 entitled Bioreactor System with Enhanced Cell Harvesting Capabilities and Related Methods, the contents of which is incorporated herein by reference in its entirety. Where possible, the description herein uses numbers corresponding to equivalent elements in the aforementioned PCT application and its Figures.

In a preferred embodiment, the mechanical action and application of same includes vibrating the bioreactor wherein the bioreactor comprises a fixed bed material with cells adhered thereto or trapped therein and wherein such cells comprise a target biomolecule. In one embodiment, mechanical action is applied to the bioreactor comprising the target biomolecule using a modified version of the system shown and described in FIGS. 2 and 2A of PCT publication WO2022/254039 entitled Bioreactor System with Enhanced Cell Harvesting Capabilities and Related Methods, the contents of which is incorporated herein by reference in its entirety. Further, the system for providing mechanical action as described hereinabove and hereinbelow is not limited to target biomolecule recovery but could readily be adapted for use for cell collection and for enabling seed train generation for small to large-scale adherent production and/or for any application that requires or benefits from use of agitating a vessel.

In an embodiment of the invention, said mechanical action or motion can be chosen from agitation, vibration, vortexing or moving a solution inside the bioreactor. In an embodiment, moving a solution comprises filling and/or draining the bioreactor with a solution. In an embodiment, said mechanical action or motion is selected from the group consisting of vibration, agitation, compaction, vortexing, rocking, applying ultrasound waves, filling and/or draining the bioreactor with a solution, expansion and combinations of the foregoing. In a preferred embodiment, the harvest can be performed under agitation. In a further embodiment, said agitation is performed at a speed of 0.5 to 2 cm/s. As such, in an embodiment, moving a liquid level relative to the fixed bed structure occurs at a speed of 0.5 to 2 cm/s. This refers to the speed of the (vertical) movement of the liquid inside the bioreactor (for instance during draining and/or filling of the bioreactor).

In an embodiment, the method of the invention comprises applying vibrations to the bioreactor. In an embodiment, said vibrations are applied during one or more of the lysis step and/or following rinsing steps. As such, it is possible to recover the biomolecules such as AAV that remain trapped in the fixed bed matrix.

In an embodiment, said vibrations are applied during one or more of the processes listed hereinabove. In an embodiment, said vibrations are applied during the rinsing step, or during the contact time between the lysis solution and the cells, or during the application of DNase or during the application of surfactant, or during the harvest of the cells or whilst using a pH below or above the isoelectric point or whilst using high conductivity solutions.

In a preferred embodiment, the cell harvest vibrating table as described in PCT/EP2022/065264 (Bioreactor System with Enhanced Cell Harvesting Capabilities and Related Methods, 3 Jun. 2022 incorporated herein by reference in its entirety) is used to harvest biomolecules such as AAV from fixed bed bioreactors. Vibrations from 20 to 100 Hz could be used in combination of filling and/or emptying cycles to transfer the vibrations to the ALI (air liquid interface) of the fixed bed structure. As such vibrations are suitable to recover fragile viable cells from a fixed bed bioreactor using vibrations and a detaching enzyme (such as trypsin), they should also be suitable for a sensitive product (such as AAV). Because detergents are used to lyse the cells, one drawback may be the creation of foam. Hence, vibrations might be more relevant after the chemical lysis in the rinsing steps with buffers that don't contain high concentrations of detergents.

As such, in an embodiment, said mechanical actions or motions are vibrations applied to said bioreactor, wherein said vibrations have a frequency from 20 to 100 Hz. In a preferred embodiment, said vibrations are performed simultaneously with moving a liquid inside the bioreactor (such as filling and/or emptying cycles to transfer the vibrations to the fixed bed structure). In an embodiment, said vibrations are applied by means of a vibrating table.

In an embodiment, the method further includes agitating the bioreactor and moving a liquid level relative to the fixed bed structure and introducing a cell lysis solution into the bioreactor. In one embodiment, the agitating and moving steps are done simultaneously. The moving step may comprise moving the liquid level higher or lower than a previous liquid level. In an embodiment, the moving step comprises at least partially draining the bioreactor of liquid, such as by moving the liquid level from adjacent a top of the fixed bed structure to adjacent a bottom of the fixed bed. The moving step may comprise adding liquid to the bioreactor, such as for example by adding a cell lysis solution to the bioreactor. The moving step may comprise moving the structure for cell entrapment/adherence and growth (the fixed bed) relative to the bioreactor to move a location of the liquid level. The liquid level may be located above the fixed bed structure prior to the moving step, which may involve raising and lowering the liquid level (such as from the top of the fixed bed structure to the bottom of the fixed bed structure, for example), a plurality of times (but it could also be only one time). Moving a liquid level relative to the fixed bed structure may also comprise using an agitator. In some embodiments, an agitator can be a rotatable, non-contact magnetic impeller, a blade or screw agitation system, or an external circulation system. In some embodiments, the agitator can comprise a disk blade turbine, a curved blade turbine, an open lade fluid foil axial impeller, a turbine impeller with pitched blades, or a three-blade propeller.

The agitating step may comprise vibrating the bioreactor. The step of introducing may comprise enzyme reagent selected from the group consisting of salt activated nuclease endonuclease and DNase.

The method may include adjusting a position of a liquid level in the fixed bed bioreactor while applying one or more additional mechanical actions to said bioreactor. In a preferred embodiment, said one or more additional mechanical actions comprise vibrating the bioreactor. The adjusting step may comprise filling and flushing the bioreactor with liquid, including by repeatedly filling and flushing the bioreactor with liquid. Adjusting a position of a liquid level in the fixed bed bioreactor may comprise using an agitator, such as an impeller, inside the bioreactor.

Still further, the method may comprise tilting the bioreactor and/or compacting a fixed bed in the bioreactor. The adjusting step may comprise moving the fixed bed relative to the bioreactor.

The method may include vibrating the bioreactor and tilting and draining the bioreactor. The vibrating, tilting, and draining steps may be performed concurrently.

See Example 2 and related FIG. 2 for data showing process steps and examples of conditions for harvest and recovery of biomolecules such as AAV cells from a fixed bed bioreactor.

In an embodiment, the invention relates to harvest of biomolecules (such as viral vectors, especially AAV) from fixed bed bioreactors in recirculation.

The inventions disclosed herein comprise processes, compositions, and devices to improve the yield and recovery of target biomolecules from cell cultures. Any step in the process and systems for implementing such systems and processes may operate in batch mode, perfusion mode, recirculation mode or combinations thereof.

The systems disclosed herein in one embodiment may comprise, for example, one or more liquid transfer devices, such as two-way or reversible pumps or other means for transmitting fluid to and from the bioreactor. The liquid transfer device may cause the fluid drained during a drain mode to be recirculated into the bioreactor during fill mode, or fresh fluid may be introduced into the bioreactor during such fill mode. The liquid transfer device may also perform only a draining, emptying, or filling cycle, and may be done using only one such cycle (e.g., one draining or filling) or multiple cycles.

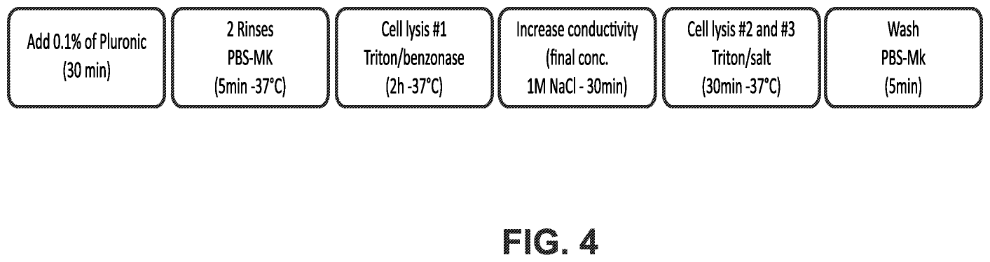

In an embodiment, target biomolecules are obtained from a fixed bed bioreactor in recirculation. In one embodiment the invention comprises recovering target biomolecules from a fixed bed bioreactor having a V/S ratio from about 0.3 to about 0.1 and a void volume of from about 10 to about 55% comprising the following steps (see also FIG. 4 for a schematic overview) and repeating one or more of the steps from 1 to 10 times, preferably from 1 to 5 times.
Add 0.1% of Pluronic (30 min)
2 rinses, PBS-MK)(5 min—37° ° C.
Cell lysis #1, Triton/benzonase (2 h—37° C.
Increase conductivity (final conc. 1M NaCl-30 min)
Cell lysis #2 and #3, Triton/salt (30 min)−37° ° C.
Wash, PBS-MK (5 min)

In one embodiment, the invention comprises a method to harvest intracellular target biomolecules, preferably AAV, from a fixed bed bioreactor including one or more of the steps described hereinabove while the bioreactor is operated in a recirculation mode. In one embodiment, the recirculation mode comprises a recirculation loop. This “artificially” increases the volume of the bioreactor allowing to reach a similar amount of enzyme and lysis buffer per surface (and then per cells and viruses) without increasing the concentration of impurities. The recirculation mode may be used throughout the entire harvesting process, it may be used intermittently, or it may be used only during one or more steps.

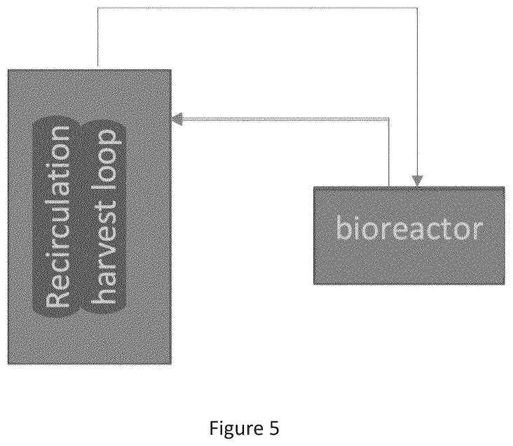

Operating the bioreactor in recirculation mode during harvesting also allows for execution of back and forth cycles (performed during the harvest, see FIG. 5) to generate mechanical actions that could improve and or increase the yield of target biomolecules, preferably AAV viruses recovered from the bioreactor. The recirculation loop may comprise a sample portion for taking samples during operation of the recirculation loop. The recirculation loop may also or alternatively be used for emptying or draining the bioreactor including during the back and forth cycles (on/off agitation cycles combined with fixed bed draining/filling). In another embodiment the recirculation loop comprises means for media exchange operation.

In one embodiment the present invention comprises one or more of the following steps to recover target biomolecules, preferably AAV viruses from a bioreactor, preferably a fixed bed bioreactor.
Perform several cycles with several rinses.
Have sufficient contact time.
Using DNase to degrade the free DNA and prevent that AAV stick to DNA adsorbed on the fixed bed fibers
Using surfactant to limit the adsorption of AAV to plastic vessel walls processes).
Perform the harvest under agitation.
Applying vibrations to the bioreactor.
Applying vibrations to the bioreactor during one or more of the lysis steps and/or following rinsing step.
Applying vibrations to the bioreactor during one or more of the processes listed hereinabove.
Operating one or more of the steps above in recirculation mode, preferably using a recirculation loop.

In an embodiment, one or more of the following steps are operated in recirculation mode, preferably using a recirculation loop: the rinsing step, the contact time between the lysis solution and the cells, the application of DNase or the application of surfactant, the harvest of the cells or whilst using a pH below or above the isoelectric point, whilst using high conductivity solutions or whilst using electrophoresis to extract one or more charged biological molecules from a fixed bed bioreactor.

See FIG. 2 for a schematic showing a preferred embodiment of the invention.

In an embodiment of the method, electrophoresis is used to extract one or more charged biological molecules from a fixed bed bioreactor. In an embodiment, an alternative electrophoresis-based solution for biological particles extraction is used.

In one embodiment the invention comprises the use of electrophoresis to extract one or more charged biological molecules from a fixed bed bioreactor, preferably from the fixed bed. Electrophoresis when applied to a fixed bed, mobilizes biological molecules within the fixed bed by the application of a constant homogenous electrical field.

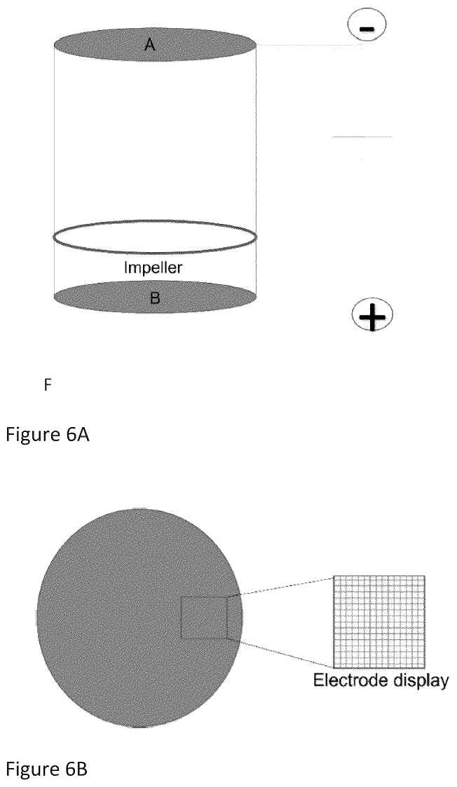

In one embodiment, the invention comprises positioning of 2 electrodes in the bioreactor (A) anode on the top of the fixed bed in direct contact with it (B) cathode on the bioreactor bottom, below the impeller (FIG. 6A). Depending on the type of biological molecule and their charge, the electrodes charge can be easily reversed.

In one embodiment the electrode structure may be similar to a net, ensuring a homogenous electric field (FIG. 6B).

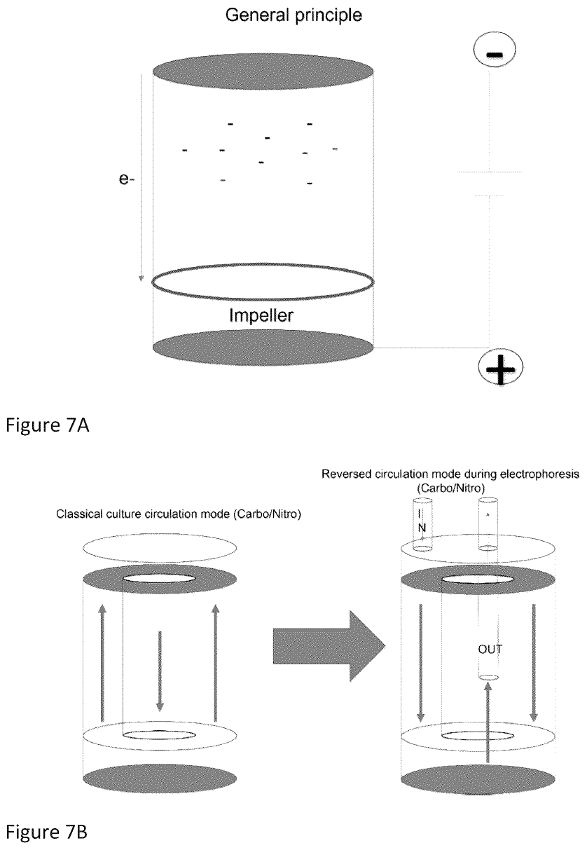

The invention is presented as a derived electrophoresis structure implemented in a fixed bed bioreactor, where the fixed bed is composed of assembled elements, such as PET FB layers or 3D structure. After the culture, a cell lysis is performed if needed, and a constant potential difference is applied between the two electrodes, resulting in the migration of any charged biomolecules (including viruses, proteins and DNA charged) (FIG. 7A).

In one embodiment, the impeller is started backward to reverse the sense of liquid circulation (FIG. 7B), in the same time that PBS buffer is added to compensate the lack of liquid within the fixed bed. As the liquid and the biological material is going to the impeller and to the bioreactor core, it is needed to pump out the excess of liquid and collect the biological product in an harvest bottle.

### Advantages:

The invention would require minimal modifications of actual bioreactor hardware and software systems, including (1) electrode implementations, and (2) adaptation in software to allow to reverse the sense of magnetic impeller.

In one embodiment the present invention comprises one or more of the following steps to recover target biomolecules, preferably AAV viruses from a bioreactor, preferably a fixed bed bioreactor.
Perform several cycles with several rinses.
Have sufficient contact time.
Use of electrophoresis to extract one or more charged biological molecules from a fixed bed bioreactor.

In one embodiment, the invention comprises a method for recovering a target biomolecule, preferably AAV, more preferably intracellular AAV by combination of detergent, Pluronic (prevent AAV adsorption), high conductivity, pH and benzonase (cleave the host cell DNA).

In a second aspect, the invention relates to a system comprising: an agitation device and a fixation system, said agitation device comprising a deck adapted for receiving a bioreactor; and said fixation system comprising at least one fastener for securing the bioreactor to the deck, and a bridge structure adapted for mounting to the bioreactor and receiving the at least one fastener. In a preferred embodiment, the system further comprises a bioreactor.

In one example, the system may include an agitation device for agitating the bioreactor and a second device for moving or changing a liquid level within the bioreactor, such as in one example by filling the bioreactor with a liquid so that the liquid level is at or above the top portion of the fixed bed and draining the bioreactor so as to move the liquid level from the fill level to the bottom of or below a bottom of the fixed bed. Alternatively or additionally, the second device may create a reciprocating, or “back and forth,” movement of a portion of the liquid between the inside and the outside of the fixed bed bioreactor. This back and forth movement of the liquid can be created by an actuator, such as one or more pumps, for creating a pulsing action where liquid is partially drained and then partially introduced into the bioreactor, or it can involve a complete draining and refilling of the bioreactor. For this purpose, the bioreactor may be associated with an inlet, an outlet or drain, each of which may be associated with suitable pumps, and a vent. The bioreactor may comprise a rigid vessel, or may comprise a disposable or single use vessel or bag.

The agitation device may be, for example, any means of applying agitating energy to the bioreactor or to the fixed bed. The portion of the bioreactor to which the energy is applied may include any part of the bioreactor as long as it results in vibration of the fixed bed. The agitation device may comprise, for example, an agitator in the form of a vibrational table, a vortex device, a shaker, or another device for applying mechanical energy to the bioreactor and/or the structure for adherent cell growth/entrapment, such as the fixed bed. The agitation device may be internal to or external to the bioreactor. The vibrating motion can be oscillating, reciprocating, or periodic, either harmonic or random. The frequency may be between 0.5 and 200 Hertz. The frequency may be 20-100 Hertz or, more specifically, 50-80 Hertz. The amplitude may be low, such as 0.5-5.0 millimeters or, more specifically, 2-3 millimeters. The second device may comprise, for example, one or more liquid transfer devices, such as two-way or reversible pumps or other means for transmitting fluid to and from the bioreactor. The second device may cause the fluid drained during a drain mode to be recirculated into the bioreactor during fill mode, or fresh fluid may be introduced into the bioreactor during such fill mode. The second device may also perform only a draining, emptying, or filling cycle, and may be done using only one such cycle (e.g., one draining or filling) or multiple cycles. The second device may be integrated with the agitation device so that these devices work in tandem or in parallel. Alternatively, the devices may be portions of a single device.

In some embodiments, a bioreactor disclosed herein can comprise a process controller. In some embodiments, the system disclosed herein can comprise one or more process controllers. In an embodiment, one or more process controllers are configured to control both the bioreactor and the system. In some embodiments, the process controller is configured to control operations of a bioreactor and/or a system and can include a plurality of sensors, a local computer, a local server, a remote computer, a remote server, or a network. In some embodiments, the bioreactor and/or the system can include one or more sensors, for example, a temperature sensor (e.g., a thermocouple), flow rate sensor, gas sensor, level sensor or any other sensor. In some embodiments, the process controller can be operational to control aspects of a biomolecule production and harvesting process, and can be coupled to sensors disposed in the bioreactor and/or the system, for example, to control the temperature, volume flow rate or gas flow rate into the bioreactor and/or the system in real time. In an embodiment, the process controller is divided in two parts, namely a Programmable Logic Controller (PLC) and a Supervisory Control and Data Acquisition (SCADA). The PLC is the intelligence of the system and is connected to the sensors and the actuators. The PLC contains only data and no power. The SCADA is important for visualization, data historian and audit trail. This SCADA system runs on a server that stores the data historians and supports the visualization. In an embodiment, information can also be visualized from a client tablet. In an embodiment, the client network can be connected directly to the server for remote access. In some embodiments, a process controller can include a Human-Machine Interface (HMI), such as a display, for example, a computer monitor, a smart phone app, a tablet app, or an analog display, that can be accessed by a user to determine the state of the system (based on the sensors comprised in the system) and to control the system by means of various actuators, such as pumps, valves, heaters and agitators. In some embodiments, the process controller can include an input, for example, a keyboard, a separated smart tablet, a key pad, a mouse, or a touch screen, to allow a user to enter control parameters for controlling the operation of the bioreactor. In some embodiments, the process controller can control access to the bioreactor.

In either case, a controller may be provided that manages the method of the current invention, where a lysis solution is added to the bioreactor, thereby lysing the cells in the bioreactor, and prior to, simultaneous with, and/or after lysing said cells, one or more mechanical actions are applied to said bioreactor, after which the one or more target biomolecules are recovered from said bioreactor. In an embodiment, a controller manages an algorithm or process for combined agitation and liquid movement back and forth to and from the bioreactor in an automated fashion or as a result of operator commands.

Using the above system, the method of the current invention can be performed, where a lysis solution is added to the bioreactor, thereby lysing the cells in the bioreactor, and prior to, simultaneous with, and/or after lysing said cells, one or more mechanical actions are applied to said bioreactor, after which the one or more target biomolecules are recovered from said bioreactor. As discussed above, said mechanical actions can include a combination of two or more mechanical actions. In a preferred embodiment, the system of the current invention allows to combine agitation to the bioreactor with movement of the liquid inside the bioreactor, in order to enhance the biomolecule harvest. For example, the system could vibrate, pulse or shake the bioreactor vessel while circulating the lysis solution via external pumping with the pulsing, or back and forth, liquid movement. Alternatively, the lysis solution can be moved internally by using internal circulation within bioreactor (such as via an agitator), or by using external recirculation—circulation or perfusion). For example, the vibration may be at a selected frequency (e.g., 20-300 Hz, including for example 60-80 Hz) and the pulsing of the liquid applied for multiple cycles (e.g., between 1 and 10, and at a flow rate of between 0.1-5 L/min). Such agitation results in a maximum energy transfer at the liquid level adjacent to the gas phase of the bioreactor. By dynamically adjusting the liquid level within the bioreactor and along the fixed bed during the vibrating/shaking/agitating, such as by using the second device (e.g., pump), the biomolecules are more effectively detached from the material of the fixed bed. Consequently, the yield or harvest of biomolecules from the bioreactor is increased in an easy and relatively inexpensive manner, and without significant added cost or complexity.

The system may further include a harvest vessel, a waste vessel, and a supply vessel containing a cell lysis solution, each of which may be in fluid communication with the bioreactor or with each other. Optional vessels for supplying rinsing and inactivation solutions may also be provided in fluid communication with the bioreactor or with any of the aforementioned vessels. Filters or other devices can be positioned between any of these vessels and/or the bioreactor. Any or all of these vessels (and solutions therein) may optionally be agitated, and may be part of a recirculation loop to allow a recirculation with the bioreactor, possibly with a reservoir. The system may also be adapted to preheat the lysis solution and/or to maintain the temperature of the cell lysis solution (usually at 37° C.). The system is designed to be used for the growth of adherent cells, as well as non-adherent cells. In an embodiment the bioreactor is a batch bioreactor. In another embodiment the bioreactor is a perfusion bioreactor. In a perfusion bioreactor equivalent volumes of media are simultaneously added to and removed from the bioreactor, while the cells are retained in the bioreactor. This provides a steady source of fresh nutrients and constant removal of cell (waste) products. Perfusion allows to attain much higher cell density and thus a higher volumetric productivity than conventional bioreactors. In addition, the perfusion bioreactor allows for secreted products to be continuously harvested during the process of removing media.

In an embodiment, the system comprises one or more devices for concentration/purification of the target biomolecule by filtration (such as tangential flow filtration (TFF), coated magnetic beads (affinity), packed bed or expanded bed chromatography, or specific purification affinity column or an ultracentrifugation steps).

In an embodiment, the system includes a concentrator. The system's concentrator can be a chosen from a number of devices known to the skilled person which are suited for reducing the volume of the liquid in which the target biomolecule resides. In some embodiments, the concentrator comprises one type of concentration device (e.g., tangential flow filter). In some embodiments, the concentrator comprises more than one type of concentration device (e.g., tangential flow filter and dead-end filter). Most of these devices are based on filtration and/or size exclusion chromatography. In one embodiment the concentrator is a filtration device, more preferably a micro-filtration device, or an ultra-filtration device or a combination of both micro- and ultra-filtration device. When the system is provided with an ultra-filtration device for reducing the volume of the liquid in which the target biomolecule resides, the membrane of the device is adapted as to allow flow through of water and low molecular weight solutes, which are in general referred to as the permeate, while macromolecules such as biomolecules are retained on the membrane in the retentate. In a further embodiment, the system is provided of a tangential flow filtration device (TFF). In an embodiment, said TFF is equipped with at least one hollow fiber having pores with a porosity sufficient to retain practically all of the target biomolecules, while permitting smaller contaminants such as growth medium and solutes to pass through the pores of the membrane. In contrast to dead-end filtration, in which the liquid is passed through a membrane or bed, and where the solids are trapped on the filter, tangential flow across the surface of the filter is allowed in the TFF device, rather than directly through the filter. Accordingly, formation of a filter cake in the TFF is avoided. In another embodiment, said TFF may be equipped with a cassette/cartridge allowing tangential flow filtration. In yet another embodiment, said TFF is a single pass tangential flow filtration (SP-TFF). This device is especially advantageous when purifying proteins such as antibodies. In some embodiments, the TFF comprises a membrane with an area of between about 1000 cm² and 2000 cm², preferably about 1500 cm². The TFF may be reused, for one time use and/or disposable. In some embodiments, the TFF is plug and play.

In an embodiment, the system is provided with one or more membrane based chromatography devices. Said membranes can occur in various form factors like columns, well plates and cassettes, with membrane volumes as small as 5.5 microliters up to several liters. Said membrane based chromatography devices include for instance protein A based affinity membrane chromatography devices for capture-step antibody purification, devices that utilize affinity membrane adsorber technologies, devices that utilize multimodal strong anion-exchange membranes, devices that utilize weak anion-exchange membranes, devices that utilize affinity membrane chromatography for selective purification of biologics that have binding affinity to sialic acid, such as sialic acid binding lectins, biomarkers and many viral surface proteins (e.g. AAV4 and AAV5) and recombinant proteins.

As described above, the invention further relates to a system for recovering target biomolecules from a fixed bed bioreactor.

In order to maintain the integrity of the bioreactor during the agitation, the bioreactor may be attached to the system and, in particular, the agitation device, using a fixation system.

The fixation system may comprise a structure for coupling the agitation device to the bioreactor, which should be sufficiently rigid in order to transfer the mechanical energy to the bioreactor.

The fixation system comprises at least one fastener for securing the bioreactor to the deck of the agitation device, and a bridge structure adapted for mounting to the bioreactor and receiving the at least one fastener. In an embodiment, said fastener comprises a strap. In a further embodiment, said fastener comprises an adjustable strap. In an embodiment, said fixation system further comprises one or more strap adaptors or guides for locating and guiding the placement of the straps.

In order to protect against mechanical damage, the fixation system should be properly fitted to the bioreactor, and should maintain and protect any fragile part of the bioreactor present (e.g., pH and DO probes) to prevent damage.

In an embodiment, the agitation device comprises a vibrating table, said vibrating table including a placeholder on which the bioreactor is to be placed. In an embodiment, the vibrating table is adapted for receiving a vessel, preferably, a bioreactor.

In an embodiment, the bridge comprises a top portion and a depending portion. The top portion as used herein is referred to as a spider portion. In an embodiment the spider portion comprises two or more strips that intersect at a common joint at the center and top of the bridge. In an embodiment, each strip comprises two legs that depend downwardly from the spider. In a preferred embodiment the strips are perpendicular to one another and intersect, the strips and legs are the same length, and the bridge is symmetrical. In an embodiment, the strips and depending portions may be any width, length and depth and such bridge structure is adaptable to fit any size bioreactor or vessel. In an embodiment, the depending portions may extend to and contact a portion of the bioreactor or the deck of the agitation device for coupling thereto. In an embodiment, the depending portions contact and are supported by a portion of the bioreactor or the deck of the agitation device.

In an embodiment, the bridge further comprises an annular portion, wherein one or more of the legs of the bridge are connected to the annular portion. The annular portion comprises a flat surface adapted for mounting the bridge to a portion of the bioreactor or the deck of the agitation device. In an embodiment, the annular portion is sized to fit to a portion of the bioreactor for mounting (or coupling) the bridge to the bioreactor. In a preferred embodiment, the annular portion is sized to fit around and mount to the lid or a portion of the lid of the bioreactor. In an embodiment, the annular portion of the bridge rests on an annular portion of the bioreactor lid. The annular portion may extend wholly or in part around the bioreactor or vessel lid. This allows for placement of the bridge to the lid of the bioreactor without removing tubes, probes, manifolds or other implements attached to the bioreactor or vessel. In an embodiment, the annular portion couples to the lid of the bioreactor. In an embodiment, the annular portion rests on a portion of the lid of the bioreactor. In an embodiment, the annular portion is located around the base of the bioreactor. The annular portion may be coupled to a portion of the bioreactor using a clipping or other mounting device.

The annular receiving portion and bridge may be coupled to the bioreactor, to one another, to a portion thereof, to the deck or combinations thereof using any technique known in the art, including but not limited to clips, latches, locking latches, and screws. In an embodiment the coupling is releasable. As such, in an embodiment, the fixation system comprises a releasable coupling.

As such, in an embodiment, the fixation system includes an annular portion part for engaging a lid or cover of the bioreactor.

In an embodiment, the agitation device comprises an agitator in the form of a vibrational table, a vortex device, a shaker, or another device for applying mechanical energy to the bioreactor and/or the fixed bed material.

In an embodiment, the system further comprises a controller for controlling the agitation device. In an embodiment, said controller controls the bioreactor or cell harvest processes.

The agitation device can be controlled manually or in an automated fashion. In an embodiment, said agitation device is remotely controlled.

The agitation device may be independently controlled, or controlled by a controller. In an embodiment, said controller is contained in a PDG box. In an embodiment, said controller may be integrally formed with the agitation device (such as for instance comprised within a vibrating table). In an embodiment the controller forms part of a docking station for the bioreactor. The docking station may include a controller having a display for displaying various parameters associated with a bioprocessing operation being conducted, and also allowing for inputs to control various aspects of the same. For instance, the docking station may include various auxiliary containers associated with pumps connected by conduits. The controller may serve to control these pumps in order to control the flow of fluid to or from the bioreactor, as well as to control mixing of the fluid in the bioreactor such as by controlling the agitation device.

In an embodiment, the bioreactor comprises a structured fixed bed bioreactor.

The fixed bed may comprise, for example, microcarriers (for instance beads manufactured from different materials like gelatin, dextran, cellulose, plastic, or glass), a structured fixed bed, a 3D printed matrix, a bed comprised of a woven or non-woven material(s), such as for example one or more sheets of such a material in direct contact or with interposed spacers, beads, hollow fibers, or any other suitable cell culture structure for promoting adherent cell growth or cell growth via entrapment. The bed may be designed in any desired shape, orientation, or form, including for example 3D porous monoliths, stacked layers (see, e.g., U.S. Pat. No. 11,111,470), parallel layers arranged vertically, layers arranged in a spiral or wound configuration, or packed beds (see, e.g., U.S. Pat. No. 8,137,959 In an embodiment, the structured fixed bed comprises a three-dimensional (3D) monolith, such as in the form of a scaffold or lattice formed of multiple interconnected units or objects. Such objects have surfaces for cell adhesion. The fixed bed may be single use in nature to avoid the cost and complexities involved in cleaning according to bioprocessing standards. Such a monolithic structured fixed bed would prevent the generation of particles (fixed bed containing PET fibers could release some free fibers), which allows for use in processes for which the product can be filtered at the end (e.g. stem cells applications of production of large viruses than cannot be sterile filtered).

Various treatments may also be applied to the fixed bed material to provide certain qualities, such as by making certain portions of the bed hydrophilic and certain portions hydrophobic, for example. Likewise, certain portions may be made cell adherent, such as for example by providing binding ligands. In a preferred embodiment, the fixed bed comprises a hydrophilic material.

In an embodiment, the fixed bed comprises a plurality of cell immobilization layers.

In an embodiment, the plurality of cell immobilization layers are arranged in a stack or a spiral configuration. In an embodiment, the cell immobilization layers are arranged either in direct contact or with a spacing between adjacent layers. In an embodiment, the cell immobilization layers are arranged with one or more spacer layers between said one or more cell immobilization layers. In an embodiment, the fixed bed is a 3D printed fixed bed.

In an embodiment, the invention relates to a system for the recovery of biomolecules from a bioreactor, said system comprising:
a bioreactor residing on a deck of an agitating device; and
a fixation system for coupling at least a portion of the bioreactor to the agitator.

In an embodiment, the system comprises a bioreactor including a structure for cell entrapment/adherence and growth, a biomolecule harvest mechanism adapted to agitate the bioreactor and to move a liquid level relative to the structure, and a vessel including a cell lysis solution in fluid communication with the bioreactor. The biomolecule harvest mechanism comprises a device for vibrating or shaking the bioreactor and/or a pump. A pump may be provided for pumping liquid so to move a liquid level relative to the structure, and a controller may be provided for controlling the pump, as well as possibly the vibrator.

In these or other embodiments, the bioreactor may be tilted relative to a horizontal plane to facilitate draining of liquid from the bioreactor. The biomolecule harvest device may comprise an actuator for moving the structure for cell entrapment/adherence and growth relative to the bioreactor to move a location of the liquid level. A controller may be provided for controlling agitating the bioreactor and move a liquid level relative to the structure for cell entrapment/adherence and growth. The controller may be adapted for controlling delivery of one or more solutions to the bioreactor.

A further aspect of the disclosure pertains to a system for harvesting cells including a bioreactor including a structure for cell entrapment/adherence and growth, an agitator adapted to agitate the bioreactor, an actuator for moving a liquid level relative to the structure for cell entrapment/adherence and growth, and a vessel including a solution in fluid communication with the bioreactor. In one embodiment, the agitator comprises a vibrator. The actuator may comprise a linear actuator and/or a pump. A controller may also be provided for controlling the actuator and/or the agitator.

In one embodiment, the structure for cell entrapment/adherence and growth comprises a fixed bed, such as a 3D printed fixed bed. The structure for cell entrapment/adherence and growth comprises a fixed bed having a plurality of cell immobilization layers, such as arranged in a stack or a spiral configuration, and either in direct contact or with a spacing between adjacent layers.

The present invention will be now described in more details, referring to examples and figures that are not limitative.

### FIGURE DESCRIPTION

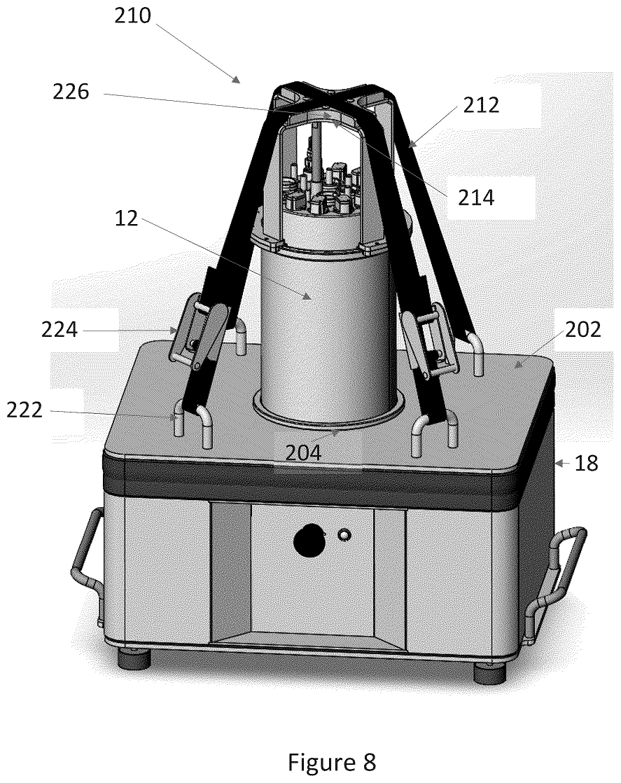

One embodiment of a system for applying mechanical action to a bioreactor is shown schematically in FIGS. 8 through 12. The system may include a device 18 for agitating the bioreactor 12. The device 18 may be, for example, any means of applying agitation energy to the bioreactor 12 or to the fixed bed (not shown). The portion of the bioreactor 12 to which the energy is applied may include any part of or portion thereof that results in vibration sufficient to cause movement of the bioreactor and or its contents including but not limited to, liquid medium, fixed bed materials, cells, cell debris and combinations thereof. The device 18 may comprise, for example, an agitator in the form of a vibrational table, a vortex device, a shaker, or another device for applying mechanical energy to the bioreactor and/or the fixed bed material. The device 18 may be internal to or external to the bioreactor 12. Preferably the device 18 is external to the bioreactor. The mechanical action, preferably a vibrating motion can be oscillating, reciprocating, or periodic, either harmonic or random.

The vibration table may comprise a motor, preferably a vibrating motor, a transformer and a frequency convertor. The frequency may be 5-100 Hertz or, more specifically, 20-100 Hertz and more specifically from between 50 and 70 Hertz. The amplitude may be low, such as 0.1-5.0 millimeters or, more specifically, 2-3 millimeters.

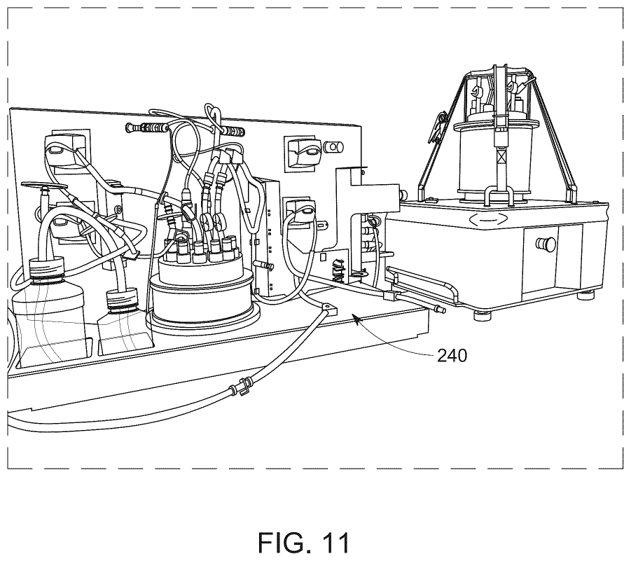

In a further embodiment, a controller (e.g., computer or processor) may be provided that manages the system and that applies mechanical action (agitation) to the bioreactor. In a further embodiment, a controller (e.g., computer or processor) with software may be provided that manages the system and the process for applying mechanical action, moving liquids associated with the various steps, combined agitation and liquid movement to and from the bioreactor 12 in an automated fashion or as a result of operator commands. See FIG. 11. In an embodiment, a single controller controls the growth of cells in the bioreactor and the recovery of cells or target biomolecule from the bioreactor using the vibration table described herein.

In one embodiment, the device used to apply mechanical action (agitation) to the bioreactor, such as a fixed bed bioreactor, may comprise a deck 202 for supporting or docking the bioreactor. In one embodiment, the deck provides an interface where mechanical energy is transferred to the bioreactor. In a further embodiment, the deck may comprise a placeholder 204 for placement of the bioreactor base or a portion thereof. The placeholder may comprise an opening, recess, insert or indentation 204 for placement of the bioreactor base or a portion thereof. In another embodiment, the deck may be replaceable/exchangeable with another deck having a different size opening, insert or indentation to accommodate a different size bioreactor or vessel. In another embodiment, a disk or ring structure may provide an interface between the deck and the bioreactor. The insert, disk or ring structure may prevent direct contact between the bioreactor and the agitation device. In an embodiment, the disk or ring is manufactured from a material comprising plastic. In a preferred embodiment the insert, disk or ring is manufactured from a material comprising polyoxymethylene (POM).

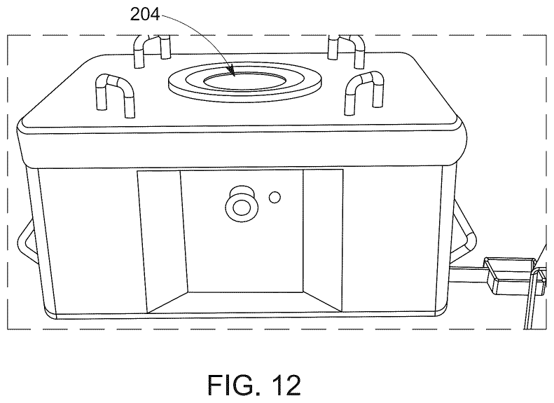

In an embodiment, the placeholder 204 (FIG. 12) comprises one or more concentric recesses to enable placement and use of different sized vessels or bioreactors. In an embodiment, the placeholder is attached to the deck of the vibration table using screws. In an embodiment, the placeholder is made from polyoxymethylene (POM) and is machined out of one plastic block.

In another embodiment, the device used to apply mechanical action (agitation) to the bioreactor may comprise a fixation system 210. The fixation system ensures an even application of force on the bioreactor by providing appropriate fixation of the bioreactor to the agitation system (i.e., vibration table) and to avoid mechanical constraints on the bioreactor, particularly the lid and seams of the bioreactor.

In an embodiment, the fixation system secures the bioreactor to the agitation device. The fixation system may minimize movement of the bioreactor, including but not limited to lateral and vertical movement of the bioreactor. The fixation system may also help to maintain the integrity of the bioreactor and minimize, reduce or prevent mechanical damage to the bioreactor that may otherwise be caused by the agitation.

In one embodiment, the fixation system may comprise a fastener to secure and/or couple at least a portion of the bioreactor directly or indirectly to the agitation device. In one embodiment, the fastener is a band or a strap 212. As used herein, the term strap is defined as a flexible material, used to fasten or secure one object to another. In one embodiment the bioreactor is fastened to the agitation device using a strap. In a further embodiment, the fixation system may comprise one or more straps. The one or more straps may be single use or reusable. The one or more straps may be adjustable, bendable, flexible, stretchable or a combination thereof. The one or more straps may be removable. The one or more straps may be made from a material comprising plastics, polymers, and blends of materials. The one or more straps may be made from a material comprising one or more of a polymer, copolymers, mixtures of polymers and mixtures of one or more polymers with a non-polymeric substance. In an embodiment, the straps may be manufactured from a material comprising one or more of rubber, nylon, neoprene, polypropylene, polyester, polyethylene, and terephthalate (PET).

In an embodiment, the strap may be a webbed material. In an embodiment, the strap may be coated. In an embodiment, the strap may be coated with a thermoplastic polyurethane (TPU) or a polyvinyl chloride (PVC) or combinations thereof. In a preferred embodiment one or more straps used in the fixation system comprise BioThane ‘BETA’® also known as BioThane Beta 520. The length, thickness and width of the one or more straps may be readily determined by one skilled in the art.

In addition to securing the bioreactor to the agitation system, the straps provide means to control how tightly or the amount of tension used in securing the bioreactor to the agitation system 18 and to distribute the pressure of the straps evenly to the bioreactor. In a preferred embodiment, the fixation system comprises two straps. In an embodiment, each strap has a first end and a second end.

In one embodiment, the fixation system comprises a bridge structure 214. The bridge structure serves to evenly apply force from the straps on the bioreactor for appropriate fixation to the vibration table. The bridge further serves to avoid mechanical constraints on certain elements of the bioreactor, particularly the lid. The bridge structure serves to protect protrusions or sensitive areas of the bioreactor from damage without blocking access thereto. The bridge in an embodiment may also provide access to ports, caps, sensors, samplers and other hardware, tubing, devices and/or manifolds that may be coupled to the bioreactor. In an embodiment, the bridge structure extends above and/or over the bioreactor lid. The bridge may be rigid or flexible. In an embodiment, the bridge is rigid. The bridge may be removable, disposable, for one time use and/or reusable. In an embodiment, the bridge is plastic. In an embodiment, the bridge is metal. In an embodiment, the bridge is made from a material comprising aluminum.

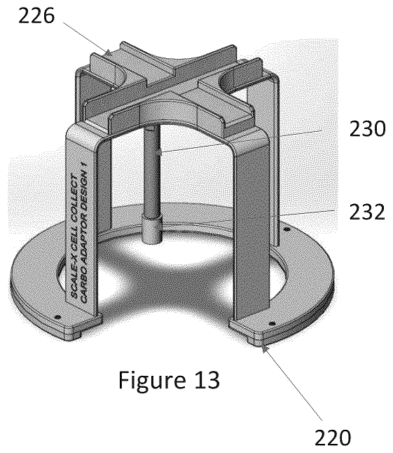

In an embodiment, the bridge comprises a top portion 214A and a depending portion 214B. The top portion as used herein is referred to as a spider portion. In an embodiment the spider portion comprises two or more strips 214A that intersect at a common joint at the center and top of the bridge. In an embodiment, each strip comprises two legs 214B that depend downwardly from the spider. In a preferred embodiment the strips are perpendicular to one another and intersect, the strips and legs are the same length, and the bridge is symmetrical. In an embodiment, the strips and depending portions may be any width, length and depth and such bridge structure is adaptable to fit any size bioreactor or vessel. In an embodiment, the depending portions may extend to and contact a portion of the bioreactor or the deck for coupling thereto. In an embodiment, the depending portions contact and are supported by a portion of the bioreactor or the deck. In an embodiment, the bridge further comprises an annular portion 214C, wherein one or more of the legs of the bridge are connected to the annular portion. The annular portion comprises a flat surface adapted for mounting the bridge to a portion of the bioreactor or the deck. The annular portion is sized to fit to a portion of the bioreactor for mounting the bridge to the bioreactor. In a preferred embodiment, the annular portion is sized to fit around and mount to the lid or a portion of the lid of the bioreactor. In an embodiment, the annular portion of the bridge rests on an annular portion of the bioreactor lid. The annular portion may extend wholly or in part around the bioreactor or vessel lid (See FIG. 13). This allows for placement of the bridge to the lid of the bioreactor without removing tubes, probes, manifolds or other implements attached to the bioreactor or vessel. In an embodiment, the annular portion couples to the lid of the bioreactor. In an embodiment, the annular portion rests on a portion of the lid of the bioreactor. In an embodiment, the annular portion is located around the base of the bioreactor. The annular portion may be coupled to a portion of the bioreactor using a clipping or other mounting device. Alternatively, the annular portion may interface with a receiving portion 220 or gasket that is mounted to the bioreactor and that is configured to receive or couple the bridge to the bioreactor or deck of the agitation device. In an embodiment, an annular receiving portion or gasket is mounted to the bioreactor lid or a portion thereof, and the annular portion of the bridge is coupled thereto or sits thereupon. In an embodiment the annular portion of the bridge further comprises a lower portion (220) or edge that is adapted to enclose a portion of the lid. This ensures appropriate fitting of the bridge to a vessel or the bioreactor lid. In an embodiment, the annular receiving portion provides an interface between the bioreactor and the metal bridge. In an embodiment the annular receiving portion prevents direct contact between the bioreactor and the bridge. In an embodiment, the annular receiving portion is manufactured from a material comprising plastic. In a preferred embodiment the annular receiving portion is manufactured from a material comprising polyoxymethylene (POM). The annular receiving portion and bridge may be coupled to the bioreactor, to one another, to a portion thereof, to the deck or combinations thereof using any technique known in the art, including but not limited to clips, latches, locking latches, and screws. In an embodiment the coupling is releasable.

In an embodiment, the fixation and agitation systems and components, may be constructed from low particle shedding materials. In an embodiment, plastic interfaces may be present in the fixation system to reduce particle shedding during use of the agitation system.

In a further embodiment, the fixation system comprises a strap guide 226 for locating and guiding the placement of the straps during installation of the straps in the fixation system. The strap guide in another embodiment serves to evenly distribute the tension provided by the straps to the bioreactor.

In another embodiment, the strap guide serves as an interface between the straps and the spider. In an embodiment, the strap guide is manufactured from a material comprising plastic. In a preferred embodiment the strap guide is manufactured from a material comprising polyoxymethylene (POM). In an embodiment, the strap guide comprises recesses for receiving and stabilizing the location of the straps. In an embodiment, the strap guide is attached to the spider. In an embodiment, the strap guide is attached to the spider using screws, adhesive welding or any method known in the art for connecting any two parts having a similar or different composition and that results in a low particle shedding structure.

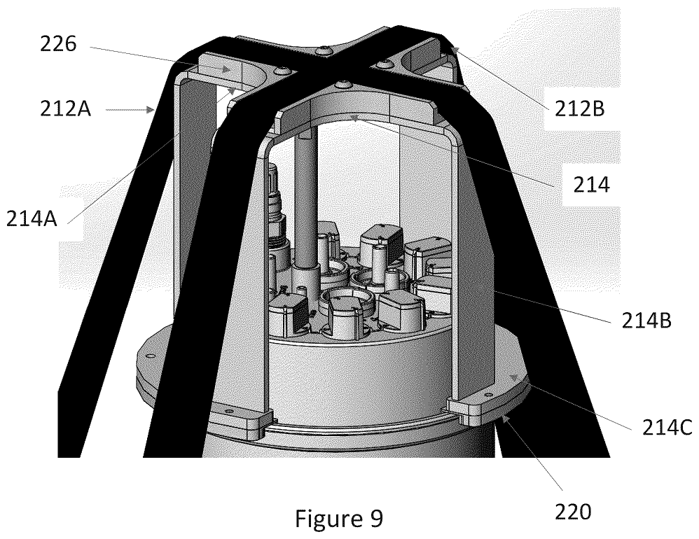

In an embodiment, the bridge further comprises a pushing pin adapted to limit displacement and/or breakage of the lid of the vessel or bioreactor. As shown in FIGS. 9 and 13, the pushing pin 230, may extend from the spider and be affixed thereto using fixation methods known in the art. The pushing pin and the bridge can be composed of the same or different materials. In an embodiment, the pushing pin and the bridge can be connected or molded (if composed out of plastic) together. In an embodiment, the pushing pin could be screwed or clipped in the bridge. In an embodiment, the pushing pin could be welded or glued onto the bridge. The pushing pin may be adjustable, removable, replaceable and/or flexible and may be manufactured of plastic, metal, rubber or combinations thereof. The pushing pin may further comprise an end cap or sleeve or cap that is made from a material such as rubber that is adapted or serves to limit displacement and breakage of the vessel or bioreactor lid. Such pushing pin when installed with the bridge will rest upon or slightly above the lid surface.

In an embodiment, the fixation system comprises anchoring devices that enable placement and fixation of the straps to the agitation system. In an embodiment the anchoring devices are anchored to the top or deck of the agitation system using any technique known in the art. In an embodiment the anchoring devices are connected to the agitation system by bolts, welding or a combination thereof. In an embodiment, the anchoring devices are constructed from metal, plastic or a combination thereof. In an embodiment, the fixation system may comprise any number of anchoring devices. In an embodiment, the anchoring devices are mounted to the agitation system. In an embodiment, the anchoring devices are mounted to the agitation system for securing and connecting the straps of the fixation system to the agitation system. In an embodiment, the anchoring devices are mounted to the deck of the agitation system. In an embodiment, the anchoring devices are hooks and the hooks are mounted to the deck or top of the agitation system. In an embodiment, there are four hooks 222. In an embodiment, the hooks are placed at or near the corners of the deck or top of the agitation system or at the corners of an imaginary square on the top of agitation device.

In an embodiment, the anchoring devices or hooks are positioned to align with the axes of the bridge and to enable positioning of the straps and fixation of the bioreactor. In an embodiment, a first end of the strap is fastened directly to an anchoring device. In an embodiment both ends of each strap of the fixation system are mounted or fastened directly to an anchoring device. In an embodiment, one or more straps are releasably coupled, fastened or secured to an anchoring device. In an embodiment, a strap of the fixation system is coupled to an anchoring device by looping the strap around the anchoring device. In an embodiment one strap is looped around two anchoring devices. In a preferred embodiment the fixation system comprises two straps and each strap is looped around or coupled to two anchoring devices. In another embodiment, the fixation system comprises means for adjusting one or more of the straps. In an embodiment the means for adjusting the strap is releasable. In an embodiment, the adjusting means is retractable. In an embodiment, adjusting comprises modifying the length of the strap. Adjustable straps ensure the bioreactor is secured to the agitation system and that an appropriate amount of tension is applied to the bioreactor by the fixation system. Further, adjustable straps provide for use of the agitation device with different sized vessels or bioreactors.

The means for adjusting the one or more straps may comprise any technique or device known in the art including stretching the strap. In another embodiment the adjusting means may comprise one or more of clamps, ratchets, buckles, latches and combinations thereof. In an embodiment, the adjusting means comprises a ratchet. In a preferred embodiment, the fixation system comprises two straps and each strap incorporates one ratchet device for adjusting the length of the strap. The ratchet device or other adjusting means may further facilitate closing of the two ends of the strap. FIG. 8 shows an embodiment of the agitation and fixation systems 18 and 210 respectively. In the embodiment shown in FIG. 8, the fixation system comprises two straps 212A and 212B. In an embodiment, each strap comprises a ratchet device 224A and 224B. Ratchet devices and assembly and implementation of same are well known in the art and the ratchet device used in the fixation system described herein is a ratchet tensioner system and preferably the ratchet tensioner system is stainless steel.

In an embodiment, a first end of a first strap is threaded into a first end of a first ratchet and the second end of the first strap is threaded into a second end of the first ratchet. Preferably there is one ratchet for each strap. Preferably, the fixation system comprises two straps wherein each strap further comprises one ratchet for adjusting the length of the strap.

As shown in FIGS. 8 and 9, in an embodiment, the fixation system comprises one end of a first strap fastened to a first hook and the second of the first strap fastened to a first end of a ratchet; a first end of a first extension strap is fastened to a second hook located diagonally to the first hook and the second end of the first strap is coupled to the second end of a ratchet and the first strap extends from the first hook along a portion of and in a first set of recesses of the bridge (also referred to as strap guides) to the first end of the ratchet, where the second end of the ratchet is fastened to the second end of the first extension strap and wherein the ratchet is adjustable. As further shown in FIGS. 8 and 9, the fixation system may further comprise a first end of a second strap fastened to a third hook and the second of the second strap fastened to a first end of a second ratchet; a first end of a second extension strap fastened to a fourth hook located diagonally to the third hook and the second end of the second strap coupled to the second end of a the second ratchet and the second strap extends from the first hook along a portion of and in the second set of recesses of the bridge (strap guides) to the first end of the second ratchet and where the second end of the second ratchet is fastened to the second end of the second extension strap and wherein the second ratchet is adjustable.

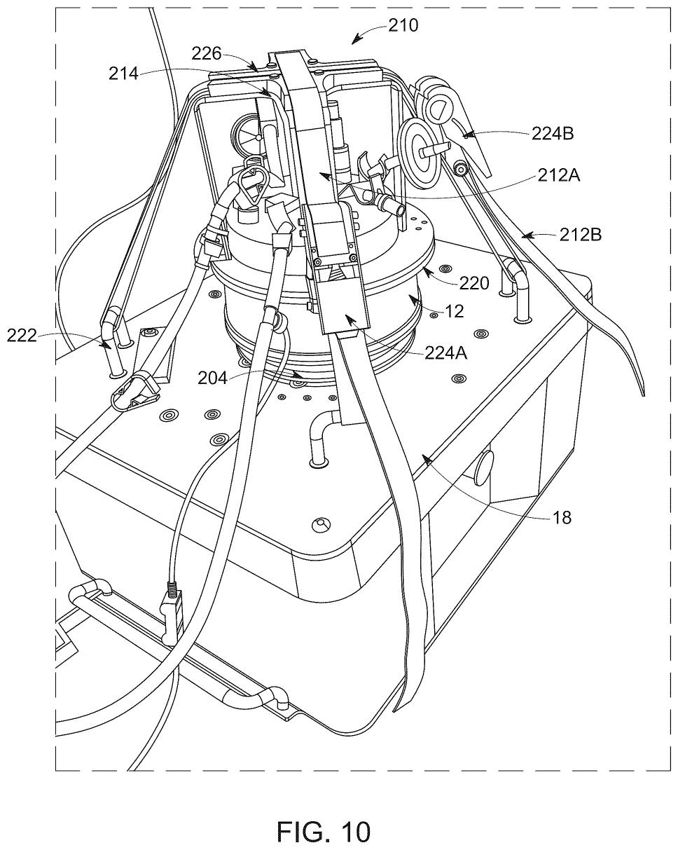

FIG. 10 shows one embodiment of a system for applying mechanical energy to a bioreactor 12 or other process related vessel, the system comprises an agitation device 18 and a fixation system 210.

In an embodiment, the bioreactor comprises a structured fixed bed and perhaps a spirally wound structured fixed bed. In an embodiment, the bioreactor comprises a cell culture, a cell culture harvest or a process solution from one or more steps of a cell harvest. In an embodiment the bioreactor comprises one or more of cells, cell debris and a target biomolecule. In an embodiment the one or more of cells, cell debris and a target biomolecule are trapped in the fixed bed, adhered to the fixed bed, or agglomerated with cells or cell debris or other biomolecules. In an embodiment, an aluminum bridge 214 comprising a spider portion 214A and four depending leg structures 214B and further comprising an annular structure portion that is mounted on top of a POM gasket 220 for receiving the annular portion of the bridge. The POM gasket and annular portion of the bridge are mounted and optionally fastened to a portion of the cap of the bioreactor. A POM strap guide 226 comprising recesses for locating and supporting the straps is mounted to the spider portion of the bridge.

A first strap is assembled by threading a first end of the strap through a first end of a first ratchet 224A; looping the free end of the strap over or under a first anchoring hook 222A; locating the strap and in a first recess in the strap guide that runs parallel to the second anchoring hook located diagonally to the first anchoring hook; looping the free end of the strap around the second anchoring hook (not shown) and bringing the end of the strap back towards the first anchoring hook by layering the strap in the strap guide over the first layer of strap and threading the end of the strap into the second end of the first ratchet; and tightening the first ratchet to the appropriate tension for operation of the agitation system. A second strap is assembled by threading a first end of the second strap through a first end of a second ratchet 224B; looping the free end of the strap over or under a third anchoring hook 222B; locating the strap and in a first recess in the strap guide that runs parallel to the fourth anchoring hook 222C located diagonally to the third anchoring hook; looping the free end of the second strap around the fourth anchoring hook and bringing the end of the strap back towards the third anchoring hook by layering the strap in the strap guide over the first layer of the second strap and threading the end of the strap into the second end of the second ratchet; and tightening the second ratchet to the appropriate tension for operation of the agitation system.

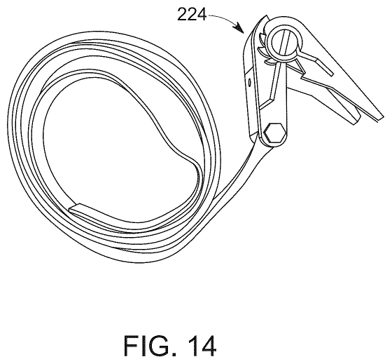

In an embodiment, the ratchet 224 is preinstalled with a strap (See FIG. 14). In another embodiment, the fixation system is installed with the bioreactor according to the following steps:
1. Place the bioreactor in the placeholder of the vibration table.
2. Place the bridge on top of the bioreactor in the orientation of the opening (adaptor engraving always facing forward on the vibrating table).
3. Be sure the center protruding pin is securely placed just parallel and left of the probes.
4. Connect the first strap around the opposing hook (on the vibration table) and over the strap guide with the lower-level of upper strap holder with the two layers of straps firmly against each other. On the other opposing hook, loop the strap around the hook with the rachet.
5. Loop the strap under the rachet hook, and pull it though, then tighten the rachet until the strap is taut. The rachet should be as close to the table as possible without touching the table in order to avoid interference with the vibrations.
6. Criss-cross the second strap in the proper holder and connect in the appropriate juxtaposing hook as in the first connected strap but place the double strap layer over the upper-level of upper strap guide.
7. Tighten until secure with the strap rachet.

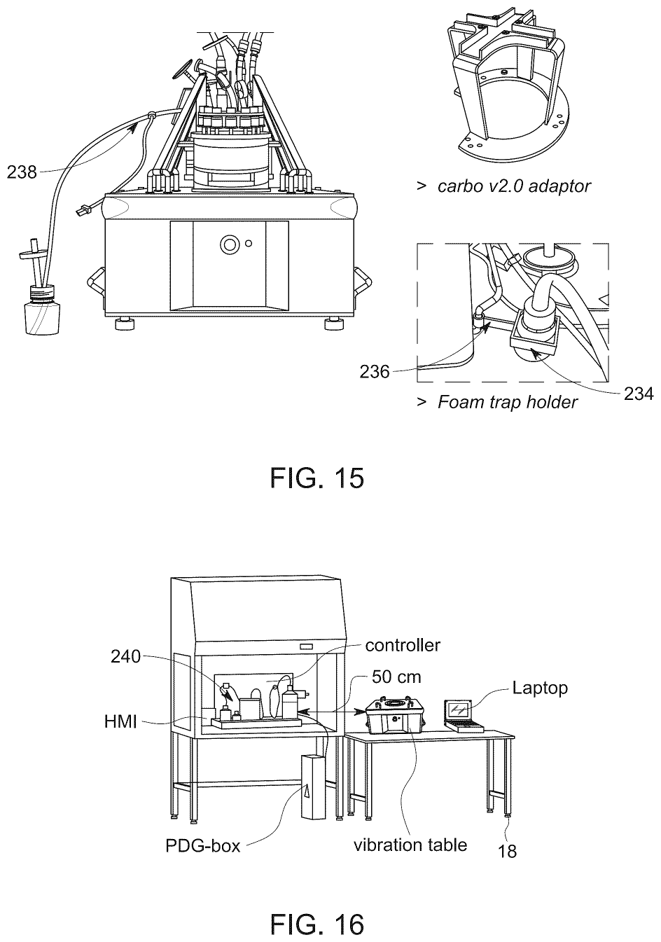

In another embodiment, the vibration table comprises a holder for securing bottles, vessels or other components during agitation of the vibration table (see FIG. 15). In an embodiment, the holder is secured to the vibrating table using a strap or arm 236. In an embodiment, the holder is reversibly secured to the vibrating table. In an embodiment, the holder is flexible, rigid and or semirigid. In an embodiment, the holder is plastic, metal, or a combination thereof. In an embodiment, the holder is manufactured from a material comprising polyoxymethylene (POM). In an embodiment the holder comprises a bracket or clamp portion (234) and a strap or arm portion (236). The strap portion secures the holder to the fixation system and the clamp or bracket portion receives a bottle or vessel thereby securing it in place and preventing it from tipping over during vibration. In an embodiment the holder receives a bottle comprising a foam trap. In an embodiment, the bottle is glass or plastic or a combination thereof. In an embodiment, the foam trap is coupled to the bioreactor.

In an embodiment, the foam trap is fluidly connected 238 to the bioreactor. In an embodiment, the foam trap is fluidly connected to the bioreactor during cell culture 240 and moves with the bioreactor when it is placed on and mounted to the vibration table (agitation system) 18 (see FIGS. 11 and 16).

In another embodiment, a pump is shared between the cell culture system 240 and the agitation system 18 to pump fluid to and from the bioreactor during cell culture growth and cell culture harvest and target biomolecule recovery.

The agitation and fixation systems described and disclosed herein and in PCT publication WO2022/254039 which is incorporated herein by reference in its entirety, may be used for cell detachment, seed trains, cell harvest and target biomolecule recovery and although the disclosed embodiments set forth that a bioreactor, optionally with a fixed bed, receives the mechanical energy from the agitation system, it is contemplated that the agitation and fixation systems may be used at any point in the production of a biomolecule and that use thereof is not limited to bioreactors but includes for example, harvest vessels and that such systems may be used with any vessel.

Non limiting examples: the examples provided herein are not intended to be limited but rather to provide working examples of the present invention. It is contemplated that all amounts and experimental variables, including parameters, concentrations, amounts and conditions could be alternatively executed in ranges above and or below those provided.

### EXAMPLES

### Example 1: Method for Obtaining AAV2 According to an Embodiment of the Invention

AAV2 is produced in a scale-X hydro bioreactor using a suspension adapted cell line. See FIG. 1 for a process flowchart of the method employed in example 1.

### Inoculation

Suspension cells are seeded from the preculture in SF (sequential fermentation) to the Scale-X bioreactor in batch mode at an equivalent cell density of 30.000 cells/cm².

### Cell Growth

After a few hours (between 4 and 24 h), the recirculation loop (containing the media required for the cell growth) is started, and cell are grown for 3 days (or 4 depending on the cell growth).

### Transfection

Cells are usually transfected when they reach 200-300.000 cells/cm². Transfection mix is prepared outside of the bioreactor and prediluted in culture media. See details below. The bioreactor should be partially emptied to be able to add the transfection complex inside the bioreactor. Transfection is performed in batch mode in a reduced volume (for instance 800 ml), stopping the alkali addition.

Change of the Loop after the Transfection

4 hours after the transfection, the recirculation loop is restarted (with fresh media in the loop partial media exchange).

### Production

Production is performed in recirculation for 3 days.

### Harvest

At the end of the run, the supernatant is harvested (extracellular AAV), the bioreactor is rinsed 2× and lysed using a lysis buffer (Pluronic F-68, Triton X-100, salt and benzonase). A washing step is performed to recover the void volume. Harvest is for instance performed at 800 ml. Harvest is performed in batch mode. See FIG. 4 for a harvest flowchart and the steps below in order to harvest the AAV.

### Harvest of Extracellular AAV

At the end of production, stop the recirculation. Add 0.1% (v/v) of Pluronic F-68 to the medium of the bioreactor and the recirculation loop and keep controls on for 30 more minutes. Empty the bioreactor vessel (void volume ˜90 mL) and collect samples of the bioreactor and the recirculation loop.

### 2× Rinses

Add (˜0.7 L) of pre-heated (37° C.) rinsing buffer to fill the bioreactor up to ˜0.8 L. The rinsing buffer comprises:
PBS
2.5 mM KCl
1 mM MgCl₂
PH: 7.0
0.1% Pluronic F-68

Apply agitation speed as 810 rpm for 5 min, then empty the bioreactor. Or apply agitation speed at 0.5-2 cm/s for 5 min, then empty the bioreactor. As such, moving the liquid level relative to the fixed bed structure occurs at a speed of 0.5 to 2 cm/s. This refers to the speed of the (vertical) movement of the liquid inside the bioreactor

Repeat the filling with rinsing buffer, mixing for 5 min and emptying step one time.

### Cell Lysis and Harvest of the AAV (Step #1)

Add ˜0.7 L of pre-heated lysis solution to the bioreactor and mix with 810 rpm for 2 h at 37° C. to reach 0.8 L. Or add pre-heated lysis solution to the bioreactor and mix at 0.5-2 cm/s for 2 h at 37° C. As such, moving a liquid level relative to the fixed bed structure occurs at a speed of 0.5 to 2 cm/s. This refers to the speed of the (vertical) movement of the liquid inside the bioreactor. Back and forth cycles are performed (every 30 min) as wash-out effect on the fixed bed helps to recover the viruses. Such back and forth cycles allow movement of a portion of the liquid between the inside and the outside of the fixed bed bioreactor (emptying/filling steps).

Lysis buffer #1 (Triton-benzonase) comprises:
1% Triton (v/v)
20 U/mL Benzonase
PBS containing 137 mM NaCl
10 mM Tris
2 mM MgCl₂
0.1% (v/v) Pluronic F-68
pH: 8.0

After 2 hours of incubation, increase the ionic strength of the lysis buffer by adjusting NaCl concentration up to 1M (final concentration) using 5M NaCl stock solution (add for instance 140 mL 5M NaCl—remove some volume inside the harvest container in order to be able to operate the bioreactor at 0.8 L). Apply agitation for 30 min, then empty the bioreactor. For instance, apply agitation speed at 810 rpm for 30 min, then empty the bioreactor. Back and forth cycles are performed (every 15 min) as wash-out effect on the fixed bed helps to recover the viruses.

### Cell Lysis and Harvest of the AAV (Step #2 and #3)

Add the pre-heated lysis solution to the bioreactor and mix with 810 rpm for 30 min at 37° C. Or add the pre-heated lysis solution to the bioreactor and start agitation for 30 min at 37° C. Back-and-forth cycles are performed (every 15 min) as wash-out effect on the fixed bed helps to recover the viruses. Repeat the filling with rinsing buffer, mixing for 30 min and emptying step one time.

Lysis buffer #2 (Triton-salt) comprises:
1% Triton (v/v)
1M NaCl
PBS
10 mM Tris
2 mM MgCl2
0.1% (v/v) Pluronic F-68
pH: 8.0

### Wash

Add 0.7 L of pre-heated wash buffer to fill the bioreactor up to ˜0.8 L.

Washing buffer comprises:
PBS
2.5 mM KCl
1 mM MgCl₂
10 mM Tris
pH: 8.0
0.1% (v/v) Pluronic F-68
   Apply agitation speed for 5 min, then empty the bioreactor. For instance, apply agitation speed as 810 rpm for 5 min, then empty the bioreactor. Repeat the filling with rinsing buffer, mixing for 5 min and emptying step one time.

### Example 2: Protocol for Harvesting rAAV2 Produced by Transfected HEK293T Cells in a Fixed Bed Bioreactor According to an Embodiment of the Invention

### Introduction

Recombinant adeno-associated virus (rAAV) is a viral vector used for gene therapy. It consists of an icosahedral capsid made of viral proteins which envelops a fragment of DNA, the transgene. The produced viral vector is used to transfer the transgene (therapeutic gene) into the nucleus of a patient's cells as a therapy to treat a life-threatening disease.

The following protocol is for the harvest of rAAV2 after the transient transfection of HEK293T cells in fixed-bed bioreactors.

The serotype 2 of rAAV is known to easily adhere to cells (membrane receptor used for transduction) or plastic material surfaces (bioreactor's walls, tubing) and to aggregate if the ionic strength is too low. Tests were carried out to investigate the affinity of rAAV2 for fixed-bed material of the bioreactor (PP and PET). Satisfactorily, no specific affinity was detected between rAAV2 and the fixed-bed material. Even if some rAAV2 are released in the supernatant (culture media) the majority remains intracellular (stocked in the cells). Fortunately, the rAAV capsids (and so rAAV2) are very resilient and physically robust; therefore, downstream processes may exploit conditions that are often avoided, such as prolonged exposure to elevated temperatures (rAAV2 is stable at 37° C.) or exposure to organic solvents. Considering the large-scale rAAV2 production in the fixed bed bioreactor, it follows that chemical lysis via the use of detergents is currently the most suitable. The objective of the harvest is to efficiently lyse the cell without affecting the integrity of the product and without promoting its aggregation or non-specific surface interactions. FIG. 2 represents the harvest flowchart. The steps and the formulation of the solutions are described herein below in the protocol description.

### Protocol Description

General remarks: Pre-warm all the solutions used to 37° C. before adding them to the bioreactor. Except the stirring and the temperature, stop the other regulations necessary for biomolecule production; maintain temperature at 37° C. and when the bioreactor is filled, keep the agitation between 0.5 and 1 cm/s. As such, moving a liquid level relative to the fixed bed structure occurs at a speed of 0.5 to 1 cm/s. This refers to the speed of the (vertical) movement of the liquid inside the bioreactor.

### Step #1—Draining of the Bioreactor

Between 30 minutes and 1 hour before draining the bioreactor, add 0.1% of Pluronic™ F-68 (v/v) in the culture supernatant. There is already Pluronic™ F-68 at 0.1% (v/v) in some culture media. Even if there is Pluronic in the culture media, it could be useful to add Pluronic™ F-68 until reaching 0.2% (v/v). Pluronic™ F-68 is a non-ionic surfactant generally used to control shear forces, prevent foam production in stirred culture and reduce cells attachment to hydrophilic surfaces. It is also known to be added to prevent AAV to stick to plastic pipets and plastic vessels during storage. Usual concentration advice for AAV is between 0.01 and 0.2%. If there is no Pluronic F-68 in the transfection mixture recirculation loop, we recommend adding it.

### Step #2—Rinsing of the Bioreactor

Use neutral solution to eliminate the residual culture medium (components that could interfere with the lysis step) and to pursue the collection of the rAAV2 extracellular fraction. Once the bioreactor is empty, fill it completely with the rinsing solution and empty it afterwards. Perform two rinsing steps because it was observed that filling/emptying the bioreactor has a positive effect on the harvest of rAAV. For this use classical PBS-MK in which Pluronic is added.

Rinsing solution formulation:
PBS
2.5 mM KCl
1 mM MgCl₂
0.1% (v/v) Pluronic
PH 7

### Step #3—Cell Lysis

This is the central step of the protocol because the majority of rAAV2 is intracellular. The contact time between the cells and the lysis solution, as well as the temperature at which the lysis is carried out, influences the efficiency of the lysis. Therefore, it is recommended to perform the lysis step for 2 hours (could depend on the processes) at 37° C. while maintaining 0.5-1 cm/s agitation. As such, moving a liquid level relative to the fixed bed structure occurs at a speed of 0.5 to 1 cm/s. This refers to the speed of the (vertical) movement of the liquid inside the bioreactor. The stirring can regularly be switched on/off to disrupt the flow. Back and forth cycles (emptying/filling steps) could be also performed as wash-out effect on the fixed-bed helps to recover the viruses. Alternatively, lysis steps could be done 2X−1 (or 2) hour each.

The lysis step could take, based on the scientific literature, between 30 min to several hours (up to 6 hours) (e.g., 4 hours) with regular sampling of the raw lysate (e.g., after 1, 2, 3, 4 hours).

The lysis buffer formulation is as follows:
1% (v/v) Tween20 or Triton X-100
1M NaCl
10 mM Tris-pH 8
0.1% Pluronic (v/v)
20-50 U/mL endonuclease (DNase) & 1-2 mM MgCl2

Tween20 or Triton X-100 is used because detergents disrupt the cell membrane. Use of detergents is concentration and time dependent, with reported concentrations of Triton X-100 between 0.1% and 0.5% and of Tween 20 between 0.1 and 1% (v/v). Zwittergent 3-14 (Calbiochem) seems also promising. Since European REACH regulation tends to ban Triton and its derivates, favor the use of Tween20 or Zwittergent. Triton is known to be more efficient than Tween. Higher concentrations of tween are recommended (Triton or Zwittergent concentration for sufficient virus release are lower compared to Tween 20. This is linked to an intrinsic property of the detergent, the critical micellar concentration).

NaCl is added because a sufficient ionic strength must be maintained to avoid rAAV2 aggregation and binding to other cellular components released during lysis. Use of a high salt concentration during the lysis step reduces rAAV2 aggregation and binding to other cellular components and reduces the surface interactions between rAAV2 and cells or cell debris that could remain adsorbed on bioreactor surfaces.

A 10 mM Tris-pH 8 buffer is used because it is observed that an alkaline solution plus appropriate amounts of detergents such as Triton X-100 or Tween20 are sufficient to lyse rAAV packaged cells and release the viral particles. Depending on the process, chemical cell lysis protocols report pH values generally between 8-9. An acidic pH (3-4) seems also promising to harvest the viral particles, but it might have an impact on the integrity of the viral proteins.

The function of 0.1% Pluronic (v/v) is similar to its function in the rinsing solution.

Nuclease treatment during the lysis step. DNase is added to:
1. Digest the host cellular nuclear material during rAAV's extraction
2. Avoid the formation of nucleic material-AAV complexes
3. Reduce the viscosity of the lysate to facilitate subsequent filtration and chromatography steps.

To limit the complexes and viscosity induced by genomic DNA, it is preferable to add the enzyme concomitantly with cell breakage. However, this step may not be compatible with the optimal conditions for enzyme activity (pH & salinity such as NaCl). In our case, if we work with the widely used Benzonase (Millipore Sigma), pH 8 doesn't alter the enzyme efficacy since it is active between 7-9 pH. However, the salt concentration is too high (1M NaCl; need to be lowered at 100-150 mM to keep an effective activity). Salt activated nuclease (SAN-HQ; ArcticZymes) can be used. Its activity at 500 mM NaCl is reported.

Preferably add between 20-50 U/ml of Benzonase during the lysis step for a minimum incubation time of 30-60 min. Also, it is necessary to add 1-2 mM MgCl₂ for the enzyme activity. Note that higher concentration of Benzonase could also be used.

A high ionic strength (NaCl>150 mM) is important to avoid viral vector aggregation, but these monovalent salts concentrations largely reduce Benzonase activity (e.g., +/−70% loss of relative activity at 150 mM NaCl). To ensure an efficient harvest, the following tradeoff can be made:

First, the lysis step begins without adding NaCl (Nota Bene: 137 mM provided by PBS buffer) to maintain an effective activity of the Benzonase (producer's brochure). As Mg²⁺ is a cofactor of Benzonase, its concentration must be accurate. Thus, we recommend the addition of 2 mM MgCl₂, this concentration being optimal for the enzyme activity. Under these salinity conditions, the Benzonase retains an effective activity and vector aggregation should be limited, but this should be carefully tested before. After 1 (or 2) hours of incubation, increase the ionic strength of the lysis buffer by adjusting NaCl concentration up to 1M. Under these conditions, Benzonase is inhibited but the risk of viral vector aggregation is reduced and/or the aggregates of virus particles solubilize (if the process is reversible). At such salinity, the virus interaction with plastic material should be reduced.

Another possibility is to conduct a separate additional step with endonuclease/Benzonase right after lysis to limit interference between cells lysis and endonuclease activity. Since the lysis step would be conducted with a high ionic strength this additional step with endonuclease should be done with a salt activated nuclease.

Nota Bene.: if endonuclease is added, it needs to be checked if inactivation is required before viral titer quantification via qPCR.

Step #4—Washing of the Bioreactor after the Cell Lysis

After 2 hours of cell lysis, conduct washes to remove the rAAV2 trapped in the fixed bed (fibers, cells debris, extracellular matrix) and/or in interaction with bioreactor's walls. Once the bioreactor is empty, fill it with the washing solution and then empty it. Repeat this step a second time. Wash two times because as mentioned in step #2 filling/emptying the bioreactor has a positive effect on the harvest of rAAV.

The washing solution formulation is as follows:
PBS
2.5 mM KCl
1 mM MgCl2
0.1% (v/v) Pluronic
10 mM Tris-pH 8

Nota Bene.: Compared to rinsing solution, washing solution pH is more alkaline

### Example 3: Exemplary Flow of Harvest and Recovery of Target Biomolecules from a Fixed Bed Bioreactor According to an Embodiment of the Invention

An exemplary flow of harvest and recovery of target biomolecules under agitation at 1 cm/s (moving a liquid level relative to the fixed bed structure at a speed of 1 cm/s) is depicted in FIG. 3 and described here below.
1. Add 0.1% of pluronic (to reach a final concentration of 0.2%) in the SN (supernatant) prior to lysis of the cells
2. Perform 2 washing steps using a wash buffer comprising 2.5 mM KCl, 1 mM MgCl₂, 0.1% pluronic and Tris pH7
3. Perform 2 lysing steps of 30 minutes each using a lysis solution comprising Triton 1%, NaCl 1M, 0.1% pluronic and Tris pH8
4. Perform 2 rinsing steps using a rinse buffer comprising 2.5 mM KCl, 1 mM MgCl₂, 0.1% pluronic and Tris pH8
5. Perform 1 lysing steps of 30 minutes using a lysis solution comprising Triton 1%, citric acid pH 3.0, 0.1% pluronic
6. Perform 2 rinsing steps using a rinse buffer comprising 2.5 mM KCl, 1 mM MgCl₂, 0.1% pluronic and Tris pH8—preheated at 37° C.
7. Add DNase (200 U/ml) for 30 minutes at 37° C. in a buffer comprising 2.5 mM KCl, 1 mM MgCl₂, 0.1% pluronic and Tris pH8
8. Perform 2 rinsing steps using a rinse buffer comprising 2.5 mM KCl, 1 mM MgCl₂, 0.1% pluronic and Tris pH8
9. Perform 1 lysing step of 30 minutes using a lysis solution comprising Triton 1%, NaCl 1M, 0.1% pluronic and Tris pH8
10. Perform 2 rinsing steps using a rinse buffer comprising 2.5 mM KCl, 1 mM MgCl₂, 0.1% pluronic and Tris pH8

As such, wash buffer is at pH 7 and rinse buffer is at pH 8.

### Example 4: Harvest of AAV from a Bioreactor Having a Fixed Bed (“Scale-X Hydro™” Bioreactor from Univercells Technologies)

An exemplary flow of harvest and recovery of AAV is depicted in FIG. 4 and described here below.

### Harvest of Extracellular AAV

At the end of production, stop the recirculation.

Add 0.1% (v/v) of Pluronic F-68 to the medium of the bioreactor and the recirculation loop and keep controls on for 30 more minutes. Empty the bioreactor vessel (void volume ˜90 mL) and collect samples of the bioreactor and the recirculation loop. Representative samples of the supernatant of the bioreactor and the recirculation loop are collected to estimate the amount of AAV present in floating cells.

### Perform 2 (or More) Rinses

Add ˜0.7 L of pre-heated (37° C.) rinsing buffer to fill the bioreactor up to ˜0.8 L.

The rinsing buffer (pH 7) comprises:
PBS
2.5 mM KCl
1 mM MgCl₂
0.1% Pluronic F-68

Apply agitation speed at 810 rpm for 5 min, then empty the bioreactor. Repeat the filling with rinsing buffer, mixing for 5 min and emptying step one time.

### Cell Lysis and Harvest of the AAV (Step #1)

Add ˜0.7 L of pre-heated lysis solution to the bioreactor and mix with 810 rpm for 2 h at 37° ° C. to reach 0.8 L. Back-and-forth cycles are performed (every 30 min) as wash-out effect on the fixed bed helps to recover the viruses.

Triton-benzonase lysis buffer #1 (pH 8) comprises:
1% Triton (v/v)
20 U/mL Benzonase
PBS containing 137 mM NaCl
10 mM Tris
2 mM MgCl₂
0.1% (v/v) Pluronic F-68

After 2 hours of incubation, increase the ionic strength of the lysis buffer by adjusting NaCl concentration up to 1M (final concentration) using 5M NaCl stock solution (add 140 ml of 5M NaCl—remove some volume inside the harvest container in order to be able to operate the bioreactor at 0.8 L). Apply agitation speed as 810 rpm for 30 min, then empty the bioreactor. Back and forth cycles are performed (every 15 min) as wash-out effect on the fixed bed helps to recover the viruses. NaCl stock solution comprises 5M NaCl.

### Cell Lysis and Harvest of the AAV (Step #2 and #3)

Add the pre-heated lysis solution to the bioreactor and mix with 810 rpm for 30 min at 37° C. Back and forth cycles are performed (every 15 min) as wash-out effect on the fixed bed helps to recover the viruses. Repeat the filling with rinsing buffer, mixing for 30 min and emptying step one time.

Triton-salt lysis buffer #2 (pH 8) comprises:
1% Triton (v/v)
1M NaCl
PBS
10 mM Tris
2 mM MgCl2
0.1% (v/v) Pluronic F-68

### Wash

Add 0.7 L of pre-heated wash buffer to fill the bioreactor up to ˜0.8 L.

Washing buffer (pH 8) comprises:
PBS
2.5 mM KCl
1 mM MgCl₂
10 mM Tris
0.1% (v/v) Pluronic F-68

Apply agitation speed as 810 rpm for 5 min, then empty the bioreactor. Repeat the filling with rinsing buffer, mixing for 5 min and emptying step one time.

### Example 5: Structured Fixed Bed Bioreactor—AAV Recovery Increased by 15% by Adding a Vibration Step

| | Total | | |
|---|---|---|---|
| Run 1 | recovered | /cm² | /cells) |
| Total AAV cells in | 4.05E+12 | | |
| supernatant | | | |
| Total intracell with | 2.20E+14 | | |
| vibration | | | |
| Total intra without | 1.91E+14 | | |
| vibration | | | |
| total AAV with vibration | 2.25E+14 | 9.36E+09 | 5.35E+04 |
| total AAV without vibration | 1.95E+14 | 8.11E+09 | 4.63E+04 |
| gain vibrations | +15% | | |
| Intra/extra with vibrations | 98% | | |
| Run 2 | Total | /cm² | /cells) |
| Total supernatant | 1.13E+13 | | |
| Total intra with vibration | 6.36E+13 | | |
| Total intra without | 3.95E+13 | | |
| vibration | | | |
| total AAV with vibration | 7.49E+13 | 3.12E+09 | 1.78E+04 |
| total AAV without vibration | 5.08E+13 | 2.12E+09 | 1.21E+04 |
| gain vibrations | +48% | | |
| Intra/extra with vibrations | 85% | | |

The present invention is in no way limited to the embodiments described in the examples and/or shown in the figures. On the contrary, methods according to the present invention may be realized in many different ways without departing from the scope of the invention.

1. A method for obtaining one or more target biomolecules from cells cultured in a fixed bed bioreactor, said method comprises:
the addition of a lysis solution to the bioreactor, thereby lysing the cells in the bioreactor, and prior to, simultaneous with, and/or after lysing said cells, applying one or more mechanical actions to said bioreactor, after which the one or more target biomolecules are recovered from said bioreactor.

2. The method according to claim 1, wherein said mechanical action is chosen from agitation, vibration, vortexing or moving a solution inside the bioreactor.

3. The method according to claim 2, wherein after said lysing of said cells, one or more washing steps are performed by filling and/or draining the bioreactor with a washing solution and/or an inactivation solution.

4. The method according to claim 2, wherein said mechanical actions are vibrations applied to said bioreactor, said vibrations have a frequency from 20 to 100 Hz.

5. The method according to claim 4, wherein said vibrations are performed simultaneously with moving a solution inside the bioreactor.

6. The method according to claim 2, wherein said vibrations are applied by means of a vibrating table.

7. The method according to claim 1, wherein said lysis solution comprises at least a detergent, such as Triton X-100, Tween 20 or Tween 80.

8. The method according to claim 1, wherein said lysis solution further comprises a DNase and/or a surfactant.

9. The method according to claim 1, wherein the method further comprises using a solution comprising 0.5 to 1 M NaCl during the lysing step and/or the washing step.

10. The method according to claim 9, wherein said solution is obtained by increasing the ionic strength of the lysis solution during the lysing step.

11. The method according to claim 1, wherein the method further comprises using a lysis solution having a pH below or above the isoelectric point of the target biomolecule.

12. A system, comprising: an agitation device (18) and a fixation system (210), said agitation device comprising a deck (202) adapted for receiving a bioreactor; and said fixation system comprising at least one fastener (212) for securing the bioreactor to the deck (202), and a bridge structure (214) adapted for mounting to the bioreactor and receiving the at least one fastener.

13. The system according to claim 12, wherein said fastener comprises an adjustable strap and wherein said fixation system further comprises one or more strap guides for locating and guiding the placement of the straps.

14. The system according to claim 12, wherein the fixation system includes an annular portion for engaging a lid or cover of the bioreactor.

15. The system according claim 12, wherein the agitation device comprises a vibrating table, said vibrating table including a placeholder on which the bioreactor is to be placed.
